(19) United States
(12) Patent Application Publication
(10) Pub. No.: US 2005/0256117 A1
(43) Pub. Date: Nov. 17, 2005

(54) Ophthalmic compositions for treating ocular hypertension

(76) Inventors: Meng Hsin Chen, Westfield, NJ (US); Luping Liu, Palainsboro, NJ (US); Peter T. Meinke, Plainfield, NJ (US); Ravi Natarajan, Scotch Plains, NJ (US); William H. Parsons, Belle Mead, NJ (US); Dong-Ming Shen, Edison, NJ (US); Min Shu, Green Brook, NJ (US); John E. Stelmach, Westfield, NJ (US); Harold B. Wood, Westfield, NJ (US); Fengqi Zhang, Edison, NJ (US); David Wisnoski, Lansdale, PA (US); James B. Doherty, Montvale, NJ (US)

Correspondence Address: MERCK AND CO., INC, P O BOX 2000, RAHWAY, NJ, 07065-0907, US

(21) Appl. No.: 10/511,636

(22) PCT Filed: Jun. 11, 2003
(86) PCT No.: PCT/US03/18413

§ 371 Date: Oct. 18, 2004

Related U.S. Application Data
(60) Provisional application No. 60/388,629, filed on Jun. 14, 2002.
(60) Provisional application No. 60/389,494, filed on Jun. 18, 2002.
(60) Provisional application No. 60/400,031, filed on Jul. 31, 2002.
(60) Provisional application No. 60/458,981, filed on Mar. 27, 2003.

(51) Int. Cl. A61K 31/5377; A61K 31/4439; A61K 31/427; C07D 417/02; C07D 413/02; C07D 43/02

(52) U.S. Cl. 514/233.5; 514/370; 548/181; 514/337; 546/270.7; 544/143

(57) ABSTRACT

This invention relates to the use of potent potassium channel blockers or a formulation thereof in the treatment of glaucoma and other conditions which leads to elevated intraoccular pressure in the eye of a patient. This invention also relates to the use of such compounds to provide a neuroprotective effect to the eye of mammalian species, particularly humans.

### BACKGROUND OF THE INVENTION

Glaucoma is a degenerative disease of the eye wherein the intraocular pressure is too high to permit normal eye function. As a result, damage may occur to the optic nerve head and result in irreversible loss of visual function. If untreated, glaucoma may eventually lead to blindness. Ocular hypertension, i.e., the condition of elevated intraocular pressure without optic nerve head damage or characteristic glaucomatous visual field defects, is now believed by the majority of ophthalmologists to represent merely the earliest phase in the onset of glaucoma.

Many of the drugs formerly used to treat glaucoma proved unsatisfactory. The early methods of treating glaucoma employed pilocarpine and produced undesirable local effects that made this drug, though valuable, unsatisfactory as a first line drug. More recently, clinicians have noted that many β-adrenergic antagonists are effective in reducing intraocular pressure. While many of these agents are effective for this purpose, there exist some patients with whom this treatment is not effective or not sufficiently effective. Many of these agents also have other characteristics, e.g., membrane stabilizing activity, that become more apparent with increased doses and render them unacceptable for chronic ocular use and can also cause cardiovascular effects.

Although pilocarpine and β-adrenergic antagonists reduce intraocular pressure, none of these drugs manifests its action by inhibiting the enzyme carbonic anhydrase, and thus they do not take advantage of reducing the contribution to aqueous humor formation made by the carbonic anhydrase pathway.

Agents referred to as carbonic anhydrase inhibitors decrease the formation of aqueous humor by inhibiting the enzyme carbonic anhydrase. While such carbonic anhydrase inhibitors are now used to treat intraocular pressure by systemic and topical routes, current therapies using these agents, particularly those using systemic routes are still not without undesirable effects. Because carbonic anhydrase inhibitors have a profound effect in altering basic physiological processes, the avoidance of a systemic route of administration serves to diminish, if not entirely eliminate, those side effects caused by inhibition of carbonic anhydrase such as metabolic acidosis, vomiting, numbness, tingling, general malaise and the like. Topically effective carbonic anhydrase inhibitors are disclosed in U.S. Pat. Nos. 4,386,098; 4,416,890; 4,426,388; 4,668,697; 4,863,922; 4,797,413; 5,378,703, 5,240,923 and 5,153,192.

Prostaglandins and prostaglandin derivatives are also known to lower intraocular pressure. U.S. Pat. No. 4,883,819 to Bito describes the use and synthesis of PGAs, PGBs and PGCs in reducing intraocular pressure. U.S. Pat. No. 4,824,857 to Goh et al. describes the use and synthesis of PGD2 and derivatives thereof in lowering intraocular pressure including derivatives wherein C-10 is replaced with nitrogen. U.S. Pat. No. 5,001,153 to Ueno et al. describes the use and synthesis of 13,14dihydro-15-keto prostaglandins and prostaglandin derivatives to lower intraocular pressure. U.S. Pat. No. 4,599,353 describes the use of eicosanoids and eicosanoid derivatives including prostaglandins and prostaglandin inhibitors in lowering intraocular pressure.

Prostaglandin and prostaglandin derivatives lower intraocular pressure by increasing uveoscleral outflow. This is true for both the F type and A type of Pgs and hence presumably also for the B, C, D, E and J types of prostaglandins and derivatives thereof. A problem with using prostaglandin derivatives to lower intraocular pressure is that these compounds often induce an initial increase in intraocular pressure, can change the color of eye pigmentation and cause proliferation of some tissues surrounding the eye.

As can be seen, there are several current therapies for treating glaucoma and elevated intraocular pressure, but the efficacy and the side effect profiles of these agents are not ideal. Recently potassium channel blockers were found to reduce intraocular pressure in the eye and therefore provide yet one more approach to the treatment of ocular hypertension and the degenerative ocular conditions related thereto. Blockage of potassium channels can diminish fluid secretion, and under some circumstances, increase smooth muscle contraction and would be expected to lower IOP and have neuroprotective effects in the eye. (see U.S. Pat. Nos. 5,573,758 and 5,925,342; Moore, et al., Invest. Ophthalmol. Vis. Sci 38, 1997; WO 89/10757, W094/28900, and WO 96/33719).

### SUMMARY OF THE INVENTION

This invention relates to the use of potent potassium channel blockers or a formulation thereof in the treatment of glaucoma and other conditions that are related to elevated intraocular pressure in the eye of a patient. This invention also relates to the use of such compounds to provide a neuroprotective effect to the eye of mammalian species, particularly humans. More particularly this invention relates to the treatment of glaucoma and/or ocular hypertension (elevated intraocular pressure) using novel indole compounds having the structural formula I: 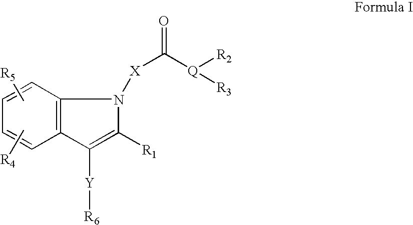
or a pharmaceutically acceptable salt, enantiomer, diastereomer or mixture thereof:
wherein,
R represents hydrogen, or C₁₋₆ alkyl;
R₁ represents hydrogen or C₁₋₆ alkyl, CF₃, C₁₋₆ alkoxy, COR^{c}, CO₂R₈, CONHCH₂CO₂R, N(R)₂, said alkyl and alkoxy optionally substituted with 1-3 groups selected from R^{b};
X represents —(CHR₇)ₚ—;
Y is not present, —CO(CH₂)ₙ—, or —CH(OR)—;
Q represents N, CR^{y}, or O, wherein R₂ is absent when Q is O;
R^{y} represents H, or C₁₋₆ alkyl;
R_{w} represents H, C₁₋₆ alkyl, —C(O)C₁₋₆ alkyl, —C(O)OC₁₋₆ alkyl, —SO₂N(R)₂, —SO₂C₁₋₆ alkyl, —SO₂C₆₋₁₀ aryl, NO₂, CN or —C(O)N(R)₂;
R₂ represents hydrogen, C₁₋₁₀ alkyl, C₁₋₆ alkylSR, —(CH₂)ₙO(CH₂)ₘOR, —(CH₂)ₙC₁₋₆ alkoxy, —(CH₂)ₙC₃₋₈ cycloalkyl, —(CH₂)ₙC₃₋₁₀ heterocyclyl, —(CH₂)ₙC₅₋₁₀ heteroaryl, —N(R)₂, —COOR, or —(CH₂)ₙC₆₋₁₀ aryl, said alkyl, heterocyclyl, aryl or heteroaryl optionally substituted with 1-3 groups selected from R^{a};
R₃ represents hydrogen, C₁₋₁₀ alkyl, —(CH₂)ₙC₃₋₈ cycloalkyl, —(CH₂)ₙC₃₋₁₀ heterocyclyl, —(CH₂)ₙC₅₋₁₀ heteroaryl, —(CH₂)ₙCOOR, —(CH₂)ₙC₆₋₁₀ aryl, —(CH₂)ₙNHR₈, —(CH₂)ₙN(R)₂, —(CH₂)ₙNHCOOR, —(CH₂)ₙN(R₈)CO₂R, —(CH₂)ₙN(R₈)COR, —(CH₂)ₙNHCOR, —(CH₂)ₙCONH(R₈), aryl, —(CH₂)ₙC₁₋₆ alkoxy, CF₃, (CH₂)ₙSO₂R, —(CH₂)ₙSO₂N(R)₂, —(CH₂)ₙCON(R)₂, —(CH₂)ₙCONHC(R)₃, —(CH₂)ₙCOR₈, nitro, cyano or halogen, said alkyl, alkoxy, heterocyclyl, aryl or heteroaryl optionally substituted with 1-3 groups of R^{a};
or, when Q is N, R₂ and R₃ taken together with the intervening N atom form a 4-10 membered heterocyclic carbon ring optionally interrupted by 1-2 atoms of O, S, C(O) or NR, and optionally having 1-4 double bonds, and optionally substituted by 1-3 groups selected from R^{a};
R⁴ and R⁵ independently represent hydrogen, C₁₋₆ alkoxy, OH, C₁₋₆ alkyl, COOR, SO₃H, O(CH₂)ₙN(R)₂, O(CH₂)ₙCO₂R, C₁₋₆ alkylcarbonyl, S(O)qR^{y}, OPO(OH)_{2,} CF₃, N(R)₂, nitro, cyano or halogen;
R₆ represents hydrogen, C₁₋₁₀ alkyl, —(CH₂)ₙC₆₋₁₀ aryl, —(CH₂)ₙC₅₋₁₀ heteroaryl, (C₆₋₁₀ aryl)O—, —(CH₂)ₙC₃₋₁₀ heterocyclyl, —(CH₂)ₙC₃₋₈ cycloalkyl, —COOR, —C(O)CO₂R, said aryl, heteroaryl, heterocyclyl and alkyl optionally substituted with 1-3 groups selected from R^{a};
R⁷ represents hydrogen, C₁₋₆ alkyl, —(CH₂)ₙCOOR or —(CH₂)ₙN(R)₂,
R⁸ represents —(CH₂)ₙC₃₋₈ cycloalkyl, —(CH₂)_{n 3-10} heterocyclyl, C₁₋₆ alkoxy or —(CH₂)ₙC₅₋₁₀ heteroaryl, said heterocyclyl, aryl or heteroaryl optionally substituted with 1-3 groups selected from R^{a};
R^{a} represents F, Cl, Br, I, CF₃, N(R)₂, NO₂, CN, —COR₈, —CONHR₈, —CON(R₈)₂, —O(CH₂)ₙCOOR, —NH(CH₂)ₙOR, —COOR, —OCF₃, —NHCOR, —SO₂R, —SO₂HR₂, —SR, (C₁-C₆ alkyl)O—, —(CH₂)ₙO(CH₂)ₘOR, —(CH₂)ₙC₁₋₆ alkoxy, (aryl)O—, —OH, (C₁-C₆ alkyl)S(Oₘ—, H₂N—C(N)—, (C₁-C₆ alkyl)C(O)-, (C₁₋₆ alkyl)OC(O)NH—, —(C₁-C₆ alkyl)NR_{w}(CH₂)ₙC₃₋₁₀ heterocyclyl-R_{w}, —(C₁-C₆ alkyl)O(CH₂)ₙC₃₋₁₀ heterocyclyl-R_{w}, —(C₁-C₆ alkyl)S(CH₂)ₙC₃₋₁₀ heterocyclyl-R_{w}, —(C₁-C₆ alkyl)-C₃₋₁₀ heterocyclyl-R_{w}, —(CH₂)ₙ-Z¹-C(=Z²)N(R)₂, —(C₂₋₆ alkenyl)NR_{w}(CH₂)ₙC₃₋₁₀ heterocyclyl-R_{w}, —(C₂₋₆ alkenyl)O(CH₂)ₙC₃₋₁₀ heterocyclyl-R_{w}, —(C₂₋₆ alkenyl)S(CH₂)ₙC₃₋₁₀ heterocyclyl-R_{w}, —(C₂₋₆ alkenyl)-C₃₋₁₀ heterocyclyl-R_{w}, —(C₂₋₆ alkenyl)-Z¹-C(=Z²)N(R)₂, —(CH₂)ₙSO₂R, —(CH₂)ₙSO₃H, —(CH₂)ₙPO(OR)₂, cyclohexyl, morpholinyl, piperidyl, pyrrolidinyl, thiophenyl, phenyl, pyridyl, imidazolyl, oxazolyl, isoxazolyl, thiazolyl, thienyl, furyl, isothiazolyl, C₂₋₆ alkenyl, and C₁-C₁₀ alkyl, said alkyl, alkenyl, alkoxy, phenyl, pyridyl, imidazolyl, oxazolyl, isoxazolyl, thiazolyl, thienyl, furyl, and isothiazolyl optionally substituted with 1-3 groups selected from C₁-C₆ alkyl, COOR, SO₃H, OH, F, Cl, Br, I, —O(CH₂)ₙCH(OH)CH₂SO₃H, and 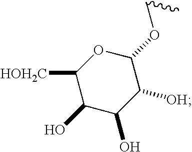
Z¹ and Z² independently represents NR_{w}, O, CH₂, or S;
R^{b} represents C₁₋₆ alkyl, —COOR, —SO₃R, —OPO(OH)₂, —(CH₂)ₙC₆₋₁₀ aryl, or —(CH₂)ₙC₅₋₁₀ heteroaryl;
R^{c} represents hydrogen, C₁₋₆ alkyl, or —(CH₂)ₙC₆₋₁₀ aryl;
m is 0-3;
n is 0-3;
q is 0-2; and
p is 0-1.

This and other aspects of the invention will be realized upon inspection of the invention as a whole.

### DETAILED DESCRIPTION OF THE INVENTION

The present invention is directed to novel potassium channel blockers of Formula I. It also relates to a method for decreasing elevated intraocular pressure or treating glaucoma by administration, preferably topical or intra-camaral administration, of a composition containing a potassium channel blocker of Formula I described hereinabove and a pharmaceutically acceptable carrier.

One embodiment of this invention is realized when X is CHR₇.

One embodiment of this invention is realized when Y is —CO(CH₂)ₙ and all other variables are as originally described. A subembodiment of this invention is realized when n is 0.

Another embodiment of this invention is realized when Y is CH(OR) and all other variables are as originally described.

Still another embodiment of this invention is realized when Q is N and all other variables are as originally described.

Still another embodiment of this invention is realized when Q is CH and all other variables are as originally described.

In another embodiment R_{w} is selected from H, C₁₋₆ alkyl, —C(O)C₁₋₆ alkyl and —C(O)N(R)₂.

Still another embodiment of this invention is realized when R₆ is (CH₂)ₙC₆₋₁₀ aryl, (CH₂)ₙC₅₋₁₀ heteroaryl, (CH₂)ₙC₃₋₁₀ heterocyclyl, or (CH₂)ₙC₃₋₈ cycloalkyl, said aryl, heteroaryl, heterocyclyl and cycloalkyl optionally substituted with 1 to 3 groups of R^{a}, and all other variables are as originally described.

Yet another embodiment of this invention is realized when R₆ is (CH₂)ₙC₆₋₁₀ aryl, (CH₂)ₙC₅₋₁₀ heteroaryl or (CH₂)ₙC₃₋₁₀ heterocyclyl, said aryl, heteroaryl and heterocyclyl optionally substituted with 1 to 3 groups of R^{a}, and all other variables are as originally described.

Yet another embodiment of this invention is realized when R⁷ is hydrogen or C₁₋₆ alkyl, and all other variables are as originally described.

Yet another embodiment of this invention is realized when Y is —CO(CH₂)ₙ, and Q is N. A subembodiment of this invention is realized when n is 0.

Still another embodiment of this invention is realized when Y is —CO(CH₂)ₙ, Q is N, R₂ is C₁₋₁₀ alkyl or C₁₋₆ alkylOH and R₃ is (CH₂)ₙC₃₋₁₀ heterocyclyl, said heterocyclyl and alkyl optionally substituted with 1 to 3 groups of R^{a}. A subembodiment of this invention is realized when n is 0.

Still another embodiment of this invention is realized when R₂ and R₃ are taken together with the intervening N atom form a 4-10 membered heterocyclic carbon ring optionally interrupted by 1-2 atoms of O, S, C(O) or NR, and optionally having 1-4 double bonds, and optionally substituted by 1-3 groups selected from R^{a}; Examples of said heterocyclic groups are: 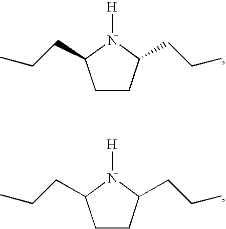
and the like.

Another embodiment of the instant invention is realized when R^{a} is selected F, Cl, Br, I, CF₃, N(R)₂, NO₂, CN, —CONHR₈, —CON(R₈)₂, —O(CH₂)ₙCOOR, —NH(CH₂)ₙOR, —COOR, —OCF₃, —NHCOR, —SO₂R, —SO₂NR₂, —SR, (C₁-C₆ alkyl)O—, —(CH₂)ₙO(CH₂)ₘOR, —(CH₂)ₙC₁₋₆ alkoxy, (aryl)O—, —OH, (C₁-C₆ alkyl)S(O)ₘ—, H₂N—C(NH)—, (C₁-C₆ alkyl)C(O)—, (C₁-C₆ alkyl)OC(O)NH—, —(C₁-C₆ alkyl)NR_{w}(CH₂)ₙC₃₋₁₀ heterocyclyl-R_{w}, —(CH₂)ₙ-Z¹-C(=Z²)N(R)₂, —(C₂₋₆ alkenyl)NR_{w}(CH₂)ₙC₃₋₁₀ heterocyclyl-R_{w},—(C₂₋₆ alkenyl)-Z¹-C(=Z²)N(R)₂,—(CH₂)ₙSO₂R, —(CH₂)ₙSO₃H, —(CH₂)ₙPO(OR)₂, C₂₋₆ alkenyl, and C₁-C₁₀ alkyl, said alkyl and alkenyl, optionally substituted with 1-3 groups selected from C₁-C₆ alkyl, and COOR;

Compounds to be used in this invention are represented by Tables 1-14

TABLE 1

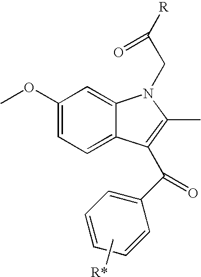

Wherein R represents:

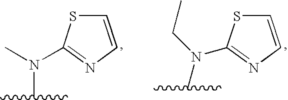

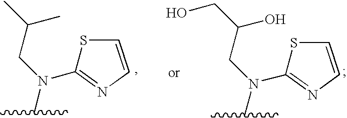

and R* represents:

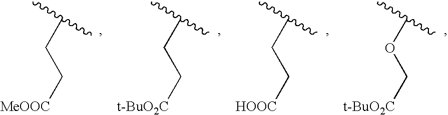

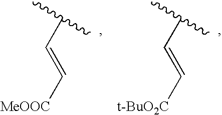

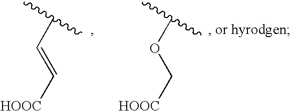

TABLE 2

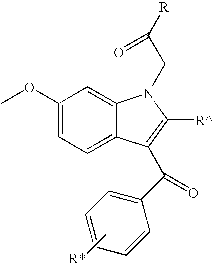

Wherein R represents:

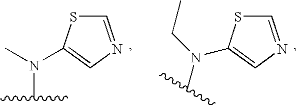

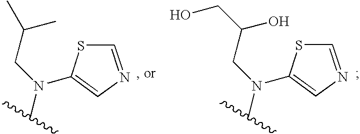

R* represents:

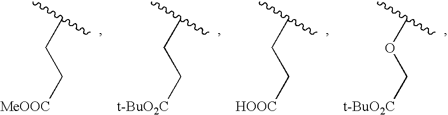

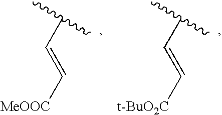

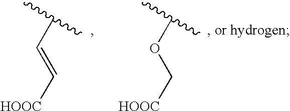

and R\hat{ } represents hydrogen or methyl

TABLE 3

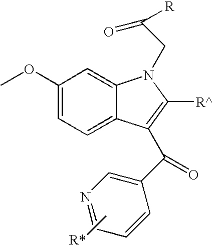

Wherein R represents:

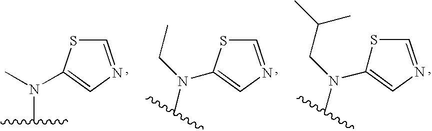

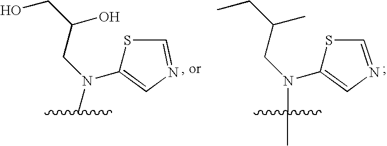

R* represents:

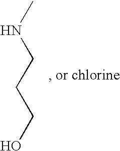

and R\hat{ } represents hydrogen or methyl;

TABLE 4

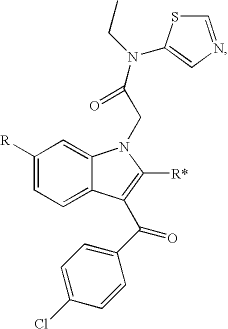

R represents methyl or methoxy and R* represents methyl or COOH;

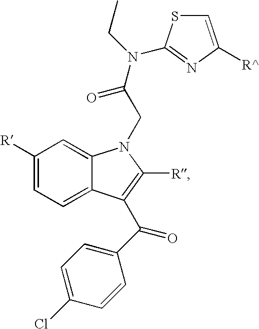

R′ represents methyl or methoxy; R\hat{ } represents hydrogen
or COOEt; R′″ represents
COOH or COOtBu; and R″ represents: COOMe, COOH, or

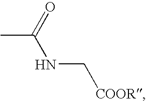

TABLE 5

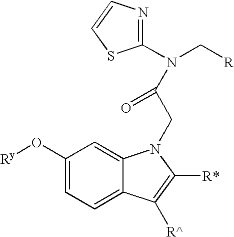

R* represents hydrogen or methyl;
R^{y} represents methyl or CF₃;
R represents methyl, (CH2)₂SCH₃,

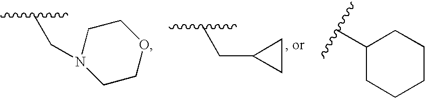

R\hat{ } represents:

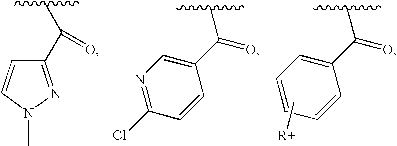

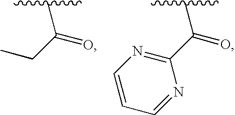

R+ represents:
(CH₂)₂CO₂H, chlorine,

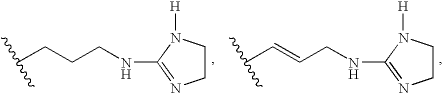

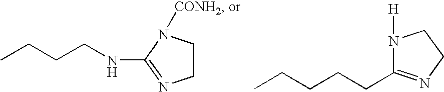

TABLE 6

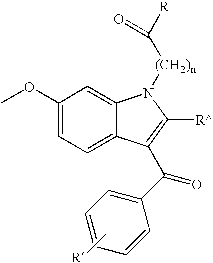

Wherein n represents 1-2;
R\hat{ } represents hydrogen or methyl
R represents:

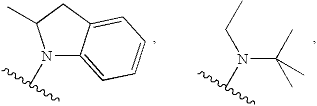

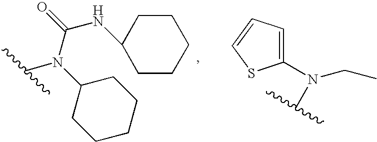

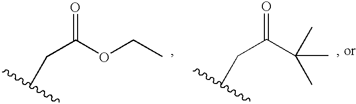

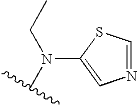

and R′ represents:
chlorine,

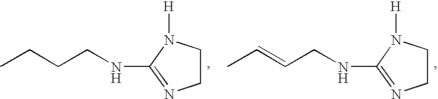

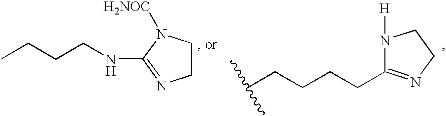

or a pharmaceutically acceptable salt, enantiomer, diastereomer or mixture thereof.

Other examples of this invention are illustrated in tables 7-14:

TABLE 7

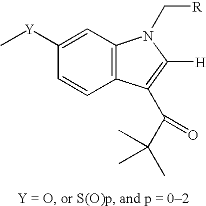

R is:

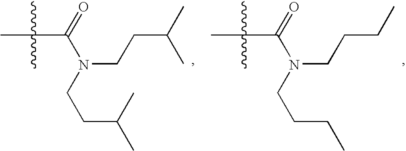

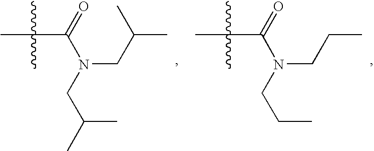

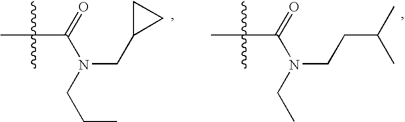

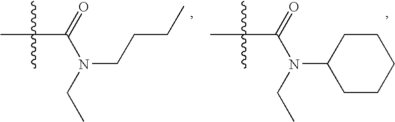

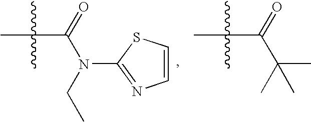

TABLE 8

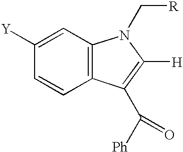

R is:

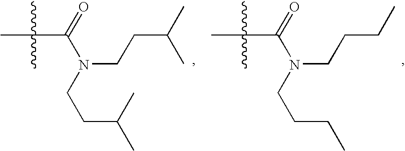

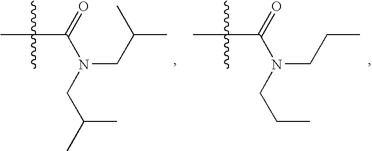

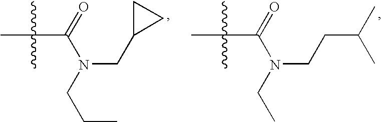

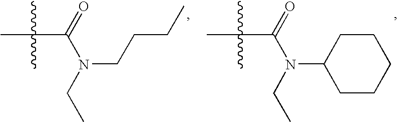

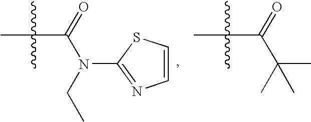

TABLE 9

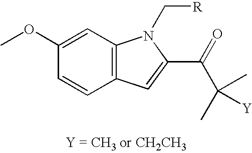

R is:

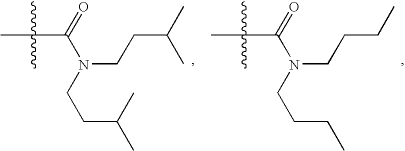

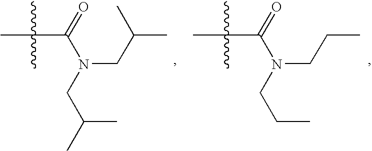

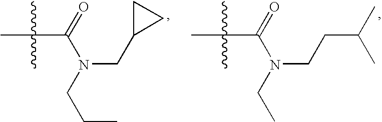

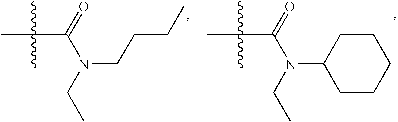

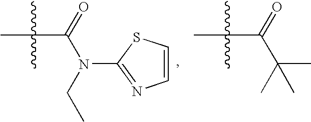

TABLE 10

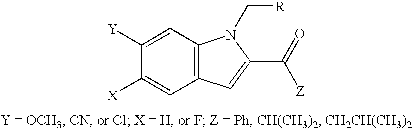

R is:

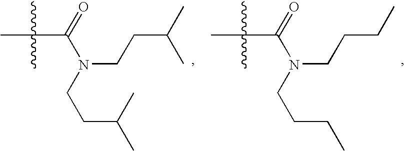

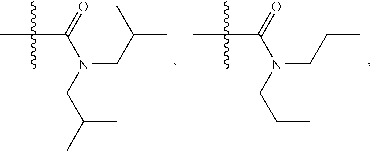

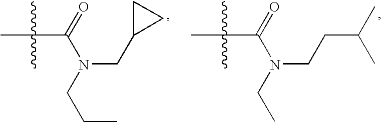

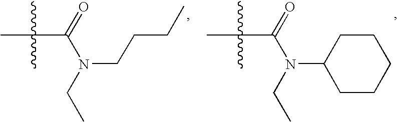

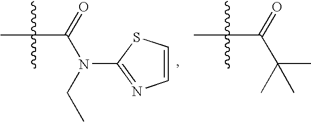

TABLE 11

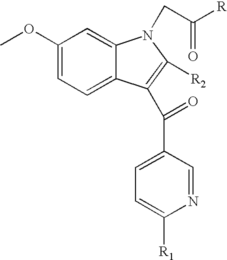

Wherein R represents:

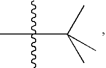

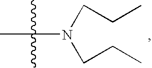

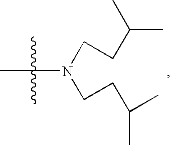

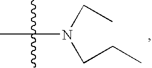

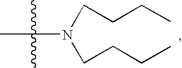

R₁ represents:

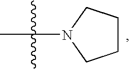

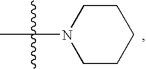

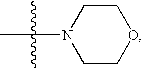

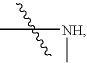

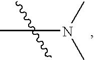

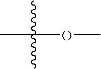

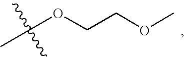

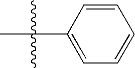

R2 represents: hydrogen or methyl or a pharmaceutically acceptable salt,
enantiomer, diastereomer or mixture thereof.

TABLE 12

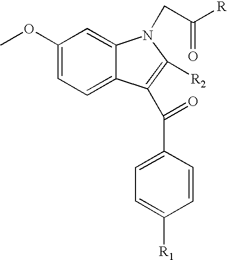

Wherein R represents:

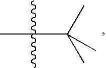

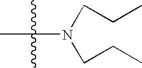

R₁ represents:

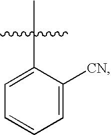

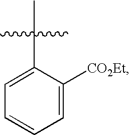

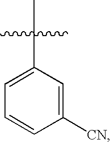

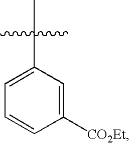

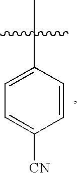

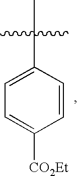

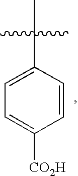

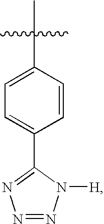

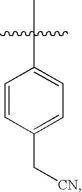

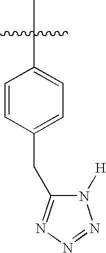

R2 represents: hydrogen or methyl

TABLE 13

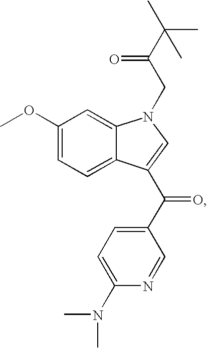

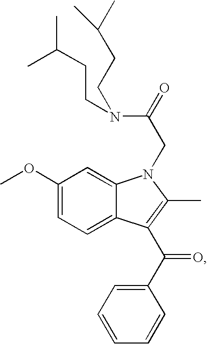

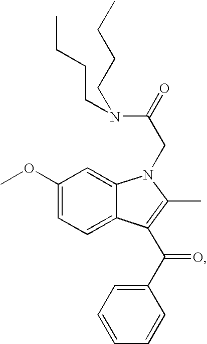

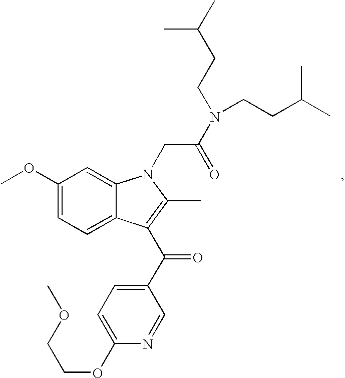

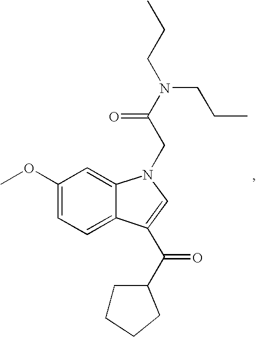

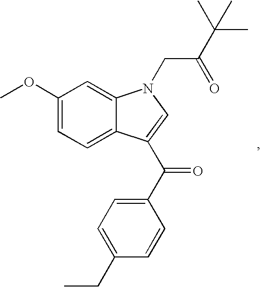

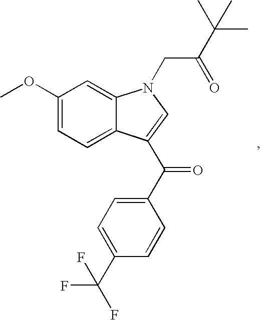

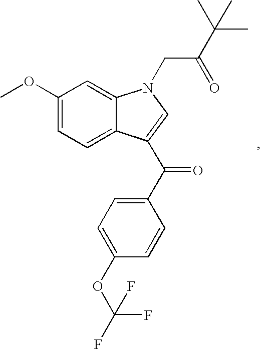

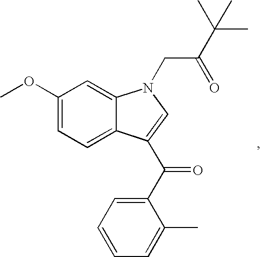

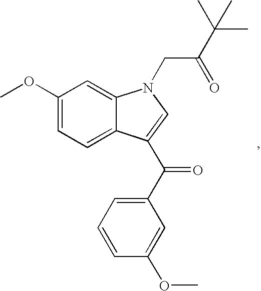

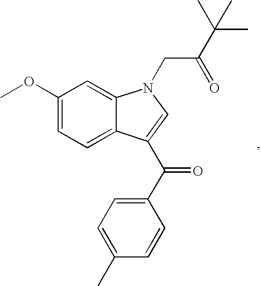

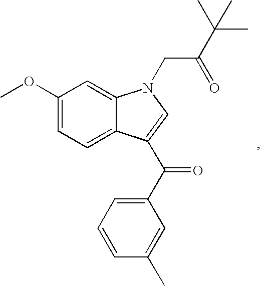

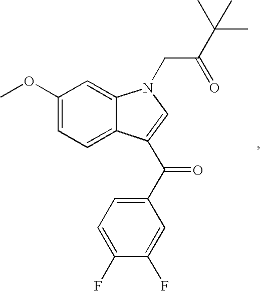

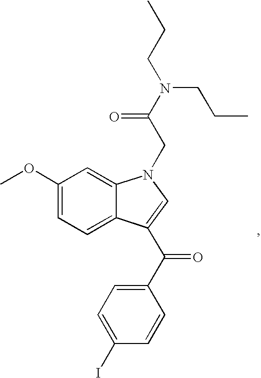

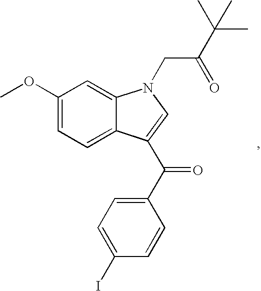

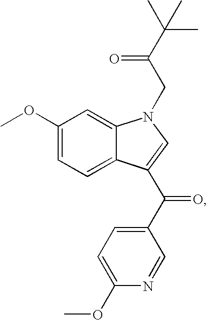

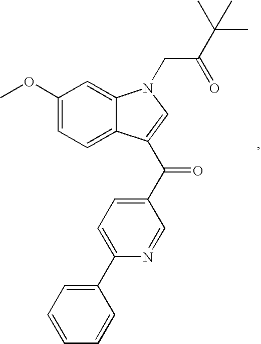

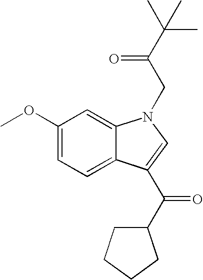

TABLE 14

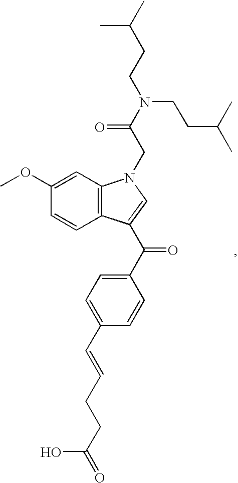

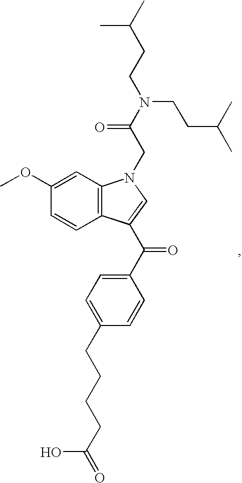

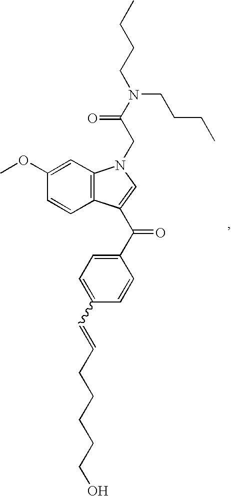

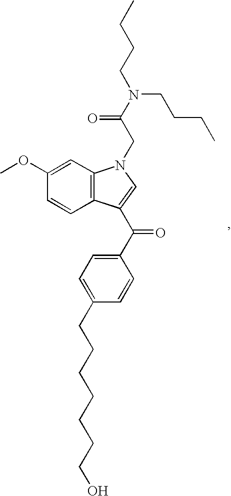

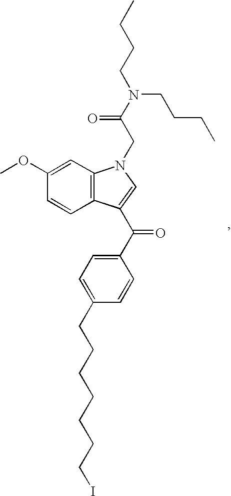

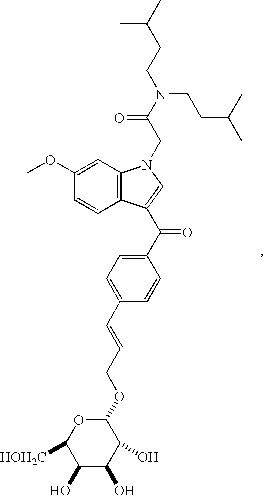

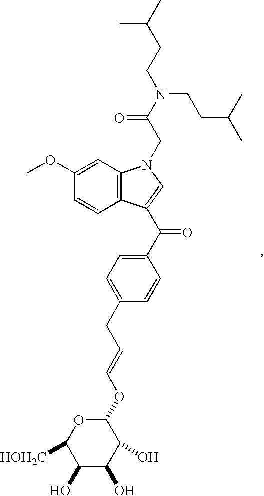

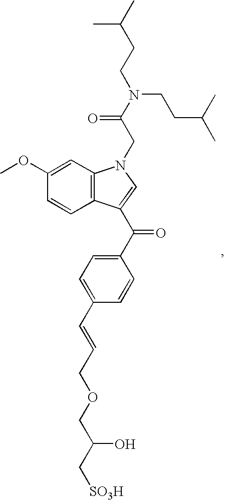

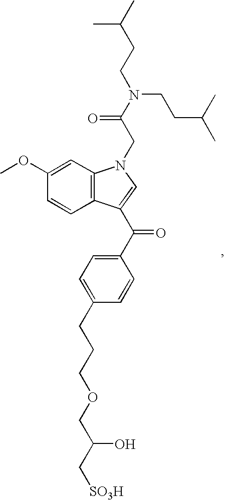

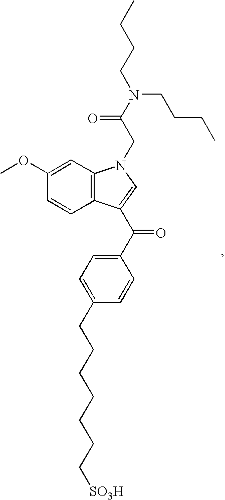

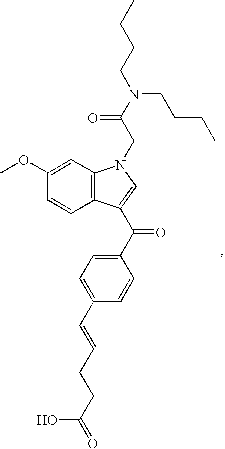

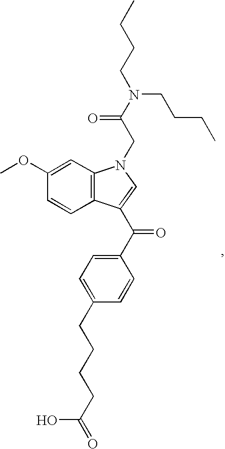

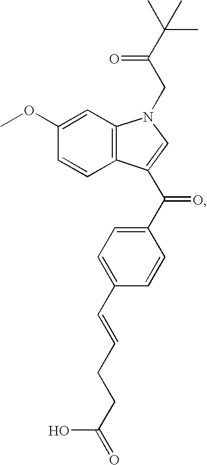

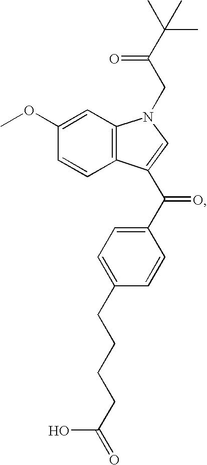

or a phannaceutically acceptable salt, enantiomer, diastereomer
or mixture thereof.

The invention is described herein in detail using the terms defined below unless otherwise specified.

The compounds of the present invention may have asymmetric centers, chiral axes and chiral planes, and occur as racemates, racemic mixtures, and as individual diastereomers, with all possible isomers, including optical isomers, being included in the present invention. (See E. L. Eliel and S. H. Wilen Stereochemistry of Carbon Compounds (John Wiley and Sons, New York 1994), in particular pages 1119-1190)

When any variable (e.g. aryl, heterocycle, R¹, R⁶ etc.) occurs more than one time in any constituent, its definition on each occurrence is independent at every other occurrence. Also, combinations of substituents/or variables are permissible only if such combinations result in stable compounds.

The term “alkyl” refers to a monovalent alkane (hydrocarbon) derived radical containing from 1 to 10 carbon atoms unless otherwise defined. It may be straight, branched or cyclic. Preferred alkyl groups include methyl, ethyl, propyl, isopropyl, butyl, t-butyl, cyclopropyl cyclopentyl and cyclohexyl. When the alkyl group is said to be substituted with an alkyl group, this is used interchangeably with “branched alkyl group”.

Cycloalkyl is a specie of alkyl containing from 3 to 15 carbon atoms, unless otherwise defined, without alternating or resonating double bonds between carbon atoms. It may contain from 1 to 4 rings, which are fused. Examples of such cycloalkyl elements include, but are not limited to, cyclopropyl, cyclobutyl, cyclopentyl, cyclohexyl and cycloheptyl.

Alkoxy refers to an alkyl group of indicated number of carbon atoms attached through an oxygen bridge, with the alkyl group optionally substituted as described herein. Said groups are those groups of the designated length in either a straight or branched configuration and if two or more carbon atoms in length, they may include a double or a triple bond. Exemplary of such alkoxy groups are methoxy, ethoxy, propoxy, isopropoxy, butoxy, isobutoxy, tertiary butoxy, pentoxy, isopentoxy, hexoxy, isohexoxy allyloxy, propargyloxy, and the like.

Halogen (halo) refers to chlorine, fluorine, iodine or bromine.

Aryl refers to aromatic rings e.g., phenyl, substituted phenyl and the like, as well as rings which are fused, e.g., naphthyl, phenanthrenyl and the like. An aryl group thus contains at least one ring having at least 6 atoms, with up to five such rings being present, containing up to 22 atoms therein, with alternating (resonating) double bonds between adjacent carbon atoms or suitable heteroatoms. Examples of aryl groups are phenyl, naphthyl, tetrahydronaphthyl, indanyl, biphenyl, phenanthryl, anthryl or acenaphthyl and phenanthrenyl, preferably phenyl, naphthyl or phenanthrenyl. Aryl groups may likewise be substituted as defined. Preferred substituted aryls include phenyl and naphthyl.

The term heterocyclyl or heterocyclic, as used herein, represents a stable 5- to 7-membered monocyclic or stable. 8- to 11-membered bicyclic heterocyclic ring which is either saturated or unsaturated, and which consists of carbon atoms and from one to four heteroatoms selected from the group consisting of N, O, and S, and including any bicyclic group in which any of the above-defined heterocyclic rings is fused to a benzene ring. The heterocyclic ring may be attached at any heteroatom or carbon atom which results in the creation of a stable structure. A fused heterocyclic ring system may include carbocyclic rings and need include only one heterocyclic ring. The term heterocycle or heterocyclic includes heteroaryl moieties. Examples of such heterocyclic elements include, but are not limited to, azepinyl, benzimidazolyl, benzisoxazolyl, benzofurazanyl, benzopyranyl, benzothiopyranyl, benzofuryl, benzothiazolyl, benzothienyl, benzoxazolyl, chromanyl, cinnolinyl, dihydrobenzofuryl, dihydrobenzothienyl, dihydrobenzothiopyranyl, dihydrobenzothiopyranyl sulfone, dihydropyrrolyl, 1,3-dioxolanyl, furyl, imidazolidinyl, imidazolinyl, imidazolyl, indolinyl, indolyl, isochromanyl, isoindolinyl, isoquinolinyl, isothiazolidinyl, isothiazolyl, isothiazolidinyl, morpholinyl, naphthyridinyl, oxadiazolyl, 2-oxoazepinyl, oxazolyl, 2-oxopiperazinyl, 2-oxopiperdinyl, 2-oxopyrrolidinyl, piperidyl, piperazinyl, pyridyl, pyrazinyl, pyrazolidinyl, pyrazolyl, pyridazinyl, pyrimidinyl, pyrrolidinyl, pyrrolyl, quinazolinyl, quinolinyl, quinoxalinyl, tetrahydrofuryl, tetrahydroisoquinolinyl, tetrahydroquinolinyl, thiamorpholinyl, thiamorpholinyl sulfoxide, thiazolyl, thiazolinyl, thienofuryl, thienothienyl, and thienyl. Preferably, heterocycle is selected from 2-azepinonyl, benzimidazolyl, 2-diazapinonyl, dihydroimidazolyl, dihydropyrrolyl, imidazolyl, 2-imidazolidinonyl, indolyl, isoquinolinyl, morpholinyl, piperidyl, piperazinyl, pyridyl, pyrrolidinyl, 2-piperidinonyl, 2-pyrimidinonyl, 2-pyrollidinonyl, quinolinyl, tetrahydrofuryl, tetrahydroisoquinolinyl, and thienyl.

The term “heteroatom” means O, S or N, selected on an independent basis.

The term “heteroaryl” refers to a monocyclic aromatic hydrocarbon group having 5 or 6 ring atoms, or a bicyclic aromatic group having 8 to 10 atoms, containing at least one heteroatom, O, S or N, in which a carbon or nitrogen atom is the point of attachment, and in which one or two additional carbon atoms is optionally replaced by a heteroatom selected from O or S, and in which from 1 to 3 additional carbon atoms are optionally replaced by nitrogen heteroatoms, said heteroaryl group being optionally substituted as described herein. Examples of such heterocyclic elements include, but are not limited to, benzimidazolyl, benzisoxazolyl, benzofurazanyl, benzopyranyl, benzothiopyranyl, benzofuryl, benzothiazolyl, benzothienyl, benzoxazolyl, chromanyl, cinnolinyl, dihydrobenzofuryl, dihydrobenzothienyl, dihydrobenzothiopyranyl, dihydrobenzothiopyranyl sulfone, furyl, imidazolyl, indolinyl, indolyl, isochromanyl, isoindolinyl, isoquinolinyl, isothiazolyl, naphthyridinyl, oxadiazolyl, pyridyl, pyrazinyl, pyrazolyl, pyridazinyl, pyrimidinyl, pyrrolyl, quinazolinyl, quinolinyl, quinoxalinyl, tetrahydroisoquinolinyl, tetrahydroquinolinyl, thiazolyl, thienofuryl, thienothienyl, thienyl and triazolyl. Additional nitrogen atoms may be present together with the first nitrogen and oxygen or sulfur, giving, e.g., thiadiazole.

This invention is also concerned with a method of treating ocular hypertension or glaucoma by administering to a patient in need thereof one of the compounds of formula I in combination with a β-adrenergic blocking agent such as timolol, a parasympathomimetic agent such as pilocarpine, carbonic anhydrase inhibitor such as dorzolamide, acetazolamide, metazolamide or brinzolamide, EP4 agonist as disclosed in U.S. Ser. No. 60/386,641, filed Jun. 6, 2002 (Attorney Docket MC059PV), 60/421,402, filed Oct. 25, 2002 (Attorney Docket MC067PV), 60/457,700, filed Mar. 26, 2003 (Attorney Docket MC080PV), 60/406,530, filed Aug. 28, 2002 (Attorney Docket MC060PV) and PCT applications PCT 02/38039, filed Nov. 27, 2002 and PCT 02/38040, filed Nov. 27, 2002, all incorporated by reference in its entirety herein, a prostaglandin such as latanoprost, rescula, S1033 or a prostaglandin derivative such as a hypotensive lipid derived from PGF2α prostaglandins. An example of a hypotensive lipid (the carboxylic acid group on the α-chain link of the basic prostaglandin structure is replaced with electrochemically neutral substituents) is that in which the carboxylic acid group is replaced with a C₁₋₆ alkoxy group such as OCH₃ (PGF₂ₐ 1-OCH₃), or a hydroxy group (PGF₂ₐ 1-OH).

Preferred potassium channel blockers are calcium activated potassium channel blockers. More preferred potassium channel blockers are high conductance, calcium activated potassium (Maxi-K) channel blockers. Maxi-K channels are a family of ion channels that are prevalent in neuronal, smooth muscle and epithelial tissues and which are gated by membrane potential and intracellular Ca²⁺.

Intraocular pressure (IOP) is controlled by aqueous humor dynamics. Aqueous humor is produced at the level of the non-pigmented ciliary epithelium and is cleared primarily via outflow through the trabecular meshwork. Aqueous humor inflow is controlled by ion transport processes. It is thought that maxi-K channels in non-pigmented ciliary epithelial cells indirectly control chloride secretion by two mechanisms; these channels maintain a hyperpolarized membrane potential (interior negative) which provides a driving force for chloride efflux from the cell, and they also provide a counter ion (K⁺) for chloride ion movement. Water moves passively with KCl allowing production of aqueous humor. Inhibition of maxi-K channels in this tissue would diminish inflow. Maxi-K channels have also been shown to control the contractility of certain smooth muscle tissues, and, in some cases, channel blockers can contract quiescent muscle, or increase the myogenic activity of spontaneously active tissue. Contraction of ciliary muscle would open the trabecular meshwork and stimulate aqueous humor outflow, as occurs with pilocarpine. Therefore maxi-K channels could profoundly influence aqueous humor dynamics in several ways; blocking this channel would decrease IOP by affecting inflow or outflow processes or by a combination of affecting both inflow/outflow processes.

The present invention is based upon the finding that maxi-K channels, if blocked, inhibit aqueous humor production by inhibiting net solute and H₂O efflux and therefore lower IOP. This finding suggests that maxi-K channel blockers are useful for treating other ophthamological dysfunctions such as macular edema and macular degeneration. It is known that lowering IOP promotes blood flow to the retina and optic nerve. Accordingly, the compounds of this invention are useful for treating macular edema and/or macular degeneration.

Macular edema is swelling within the retina within the critically important central visual zone at the posterior pole of the eye. An accumulation of fluid within the retina tends to detach the neural elements from one another and from their local blood supply, creating a dormancy of visual function in the area.

Glaucoma is characterized by progressive atrophy of the optic nerve and is frequently associated with elevated intraocular pressure (IOP). It is possible to treat glaucoma, however, without necessarily affecting IOP by using drugs that impart a neuroprotective effect. See Arch. Ophthalmol. Vol. 112, January 1994, pp. 37-44; Investigative Ophthamol. & Visual Science, 32, 5, April 1991, pp. 1593-99. It is believed that maxi-K channel blockers which lower IOP are useful for providing a neuroprotective effect. They are also believed to be effective for increasing retinal and optic nerve head blood velocity and increasing retinal and optic nerve oxygen by lowering IOP, which when coupled together benefits optic nerve health. As a result, this invention further relates to a method for increasing retinal and optic nerve head blood velocity, increasing retinal and optic nerve oxygen tension as well as providing a neuroprotective effect or a combination thereof.

As indicated above, potassium channel antagonists are useful for a number of physiological disorders in mammals, including humans. Ion channels, including potassium channels, are found in all mammalian cells and are involved in the modulation of various physiological processes and normal cellular homeostasis. Potassium ions generally control the resting membrane potential, and the efflux of potassium ions causes repolarization of the plasma membrane after cell depolarization. Potassium channel antagonists prevent repolarization and enable the cell to stay in the depolarized, excited state.

There are a number of different potassium channel subtypes. Physiologically, one of the most important potassium channel subtypes is the Maxi-K channel which is present in neuronal tissue, smooth muscle and epithelial tissue. Intracellular calcium concentration (Ca²⁺ᵢ) and membrane potential gate these channels. For example, Maxi-K channels are opened to enable efflux of potassium ions by an increase in the intracellular Ca²⁺ concentration or by membrane depolarization (change in potential). Elevation of intracellular calcium concentration is required for neurotransmitter release. Modulation of Maxi-K channel activity therefore affects transmitter release from the nerve terminal by controlling membrane potential, which in turn affects the influx of extracellular Ca²⁺ through voltage-gated calcium channels. The compounds of the present invention are therefore useful in the treatment of neurological disorders in which neurotransmitter release is impaired.

A number of marketed drugs function as potassium channel antagonists. The most important of these include the compounds Glyburide, Glipizide and Tolbutamide. These potassium channel antagonists are useful as antidiabetic agents. The compounds of this invention may be combined with one or more of these compounds to treat diabetes.

Potassium channel antagonists are also utilized as Class 3 antiarrhythmic agents and to treat acute infarctions in humans. A number of naturally occuring toxins are known to block potassium channels including Apamin, Iberiotoxin, Charybdotoxin, Noxiustoxin, Kaliotoxin, Dendrotoxin(s), mast cell degranuating (MCD) peptide, and β-Bungarotoxin (β-BTX). The compounds of this invention may be combined with one or more of these compounds to treat arrhythmias.

Depression is related to a decrease in neurotransmitter release. Current treatments of depression include blockers of neurotransmitter uptake, and inhibitors of enzymes involved in neurotransmitter degradation which act to prolong the lifetime of neurotransmitters.

Alzheimer's disease is also characterized by a diminished neurotransmitter release. Alzheimer's disease is a neurodegenerative disease of the brain leading to severely impaired cognition and functionality. This disease leads to progressive regression of memory and learned functions. Alzheimer's disease is a complex disease that affects cholinergic neurons, as well as serotonergic, noradrenergic and other central neurotransmitter systems. Manifestations of Alzheimer's disease extend beyond memory loss and include personality changes, neuromuscular changes, seizures, and occasionally psychotic features.

Alzheimer's disease is the most common type of dementia in the United States. Some estimates suggest that up to 47% of those older than 85 years have Alzheimer's disease. Since the average age of the population is on the increase, the frequency of Alzheimer's disease is increasing and requires urgent attention. Alzheimer's is a difficult medical problem because there are presently no adequate methods available for its prevention or treatment.

Three classes of drugs are being investigated for the treatment of Alzheimer's disease. The first class consists of compounds that augment acetylcholine neurotransmitter function. Currently, cholinergic potentiators such as the anticholinesterase drugs are being used in the treatment of Alzheimer's disease. In particular, physostigmine (eserine), an inhibitor of acetylcholinesterase, has been used in its treatment. The administration of physostigmine has the drawback of being considerably limited by its short half-life of effect, poor oral bioavailability, and severe dose-limiting side-effects, particularly towards the digestive system. Tacrine (tetrahydroaminocridine) is another cholinesterase inhibitor that has been employed; however, this compound may cause hepatotoxicity.

A second class of drugs that are being investigated for the treatment of Alzheimer's disease is nootropics that affect neuron metabolism with little effect elsewhere. These drugs improve nerve cell function by increasing neuron metabolic activity. Piracetam is a nootropic that may be useful in combination with acetylcholine precursors and may benefit Alzheimer's patients who retain some quantity of functional acetylcholine release in neurons. Oxiracetam is another related drug that has been investigated for Alzheimer treatment.

A third class of drugs is those drugs that affect brain vasculature. A mixture of ergoloid mesylates is used for the treatment of dementia. Ergoloid mesylates decrease vascular resistance and thereby increase cerebral blood flow. Also employed are calcium channel blocking drugs including Nimodipine which is a selective calcium channel blocker that affects primarily brain vasculature.

Other miscellaneous drugs are targeted to modify other defects found in Alzheimer's disease. Selegiline, a monoamine oxidase B inhibitor, which increases brain dopamine and norepinephrine has reportedly caused mild improvement in some Alzheimer's patients. Aluminum chelating agents have been of interest to those who believe Alzheimer's disease is due to aluminum toxicity. Drugs that affect behavior, including neuroleptics, and anxiolytics have been employed. Side effects of neuroleptics range from drowsiness and anti cholinergic effects to extrapyramidal side effects; other side effects of these drugs include seizures, inappropriate secretion of antidiuretic hormone, jaundice, weight gain and increased confusion. Anxiolytics, which are mild tranquilizers, are less effective than neuroleptics, but also have milder side effects. Use of these behavior-affecting drugs, however, remains controversial. The present invention is related to novel compounds which are useful as potassium channel antagonists. It is believed that certain diseases such as depression, memory disorders and Alzheimers disease are the result of an impairment in neurotransmitter release. The potassium channel antagonists of the present invention may therefore be utilized as cell excitants which should stimulate an unspecific release of neurotransmitters such as acetylcholine, serotonin and dopamine. Enhanced neurotransmitter release should reverse the symptoms associated with depression and Alzheimers disease.

The compounds within the scope of the present invention exhibit potassium channel antagonist activity and thus are useful in disorders associated with potassium channel malfunction. A number of cognitive disorders such as Alzheimer's Disease, memory loss or depression may benefit from enhanced release of neurotransmitters such as serotonin, dopamine or acetylcholine and the like. Blockage of Maxi-K channels maintains cellular depolarization and therefore enhances secretion of these vital neurotransmitters.

The compounds of this invention may be combined with anticholinesterase drugs such as physostigmine (eserine) and Tacrine (tetrahydroaminocridine), nootropics such as Piracetam, Oxiracetam, ergoloid mesylates, selective calcium channel blockers such as Nimodipine, or monoamine oxidase B inhibitors such as Selegiline, in the treatment of Alzheimer's disease. The compounds of this invention may also be combined with Apamin, Iberiotoxin, Charybdotoxin, Noxiustoxin, Kaliotoxin, Dendrotoxin(s), mast cell degranuating (MCD) peptide, β-Bungarotoxin (β-BTX) or a combination thereof in treating arrythmias. The compounds of this invention may further be combined with Glyburide, Glipizide, Tolbutamide or a combination thereof to treat diabetes.

The herein examples illustrate but do not limit the claimed invention. Each of the claimed compounds are potassium channel antagonists and are thus useful in the decribed neurological disorders in which it is desirable to maintain the cell in a depolarized state to achieve maximal neurotransmitter release. The compounds produced in the present invention are readily combined with suitable and known pharmaceutically acceptable excipients to produce compositions which may be administered to mammals, including humans, to achieve effective potassium channel blockage.

For use in medicine, the salts of the compounds of formula I will be pharmaceutically acceptable salts. Other salts may, however, be useful in the preparation of the compounds according to the invention or of their pharmaceutically acceptable salts. When the compound of the present invention is acidic, suitable “pharmaceutically acceptable salts” refers to salts prepared form pharmaceutically acceptable non-toxic bases including inorganic bases and organic bases. Salts derived, from inorganic bases include aluminum, ammonium, calcium, copper, ferric, ferrous, lithium, magnesium, manganic salts, manganous, potassium, sodium, zinc and the like. Particularly preferred are the ammonium, calcium, magnesium, potassium and sodium salts. Salts derived from pharmaceutically acceptable organic non-toxic bases include salts of primary, secondary and tertiary amines, substituted amines including naturally occurring substituted amines, cyclic amines and basic ion exchange resins, such as arginine, betaine caffeine, choline, N,N¹-dibenzylethylenediamine, diethylamin, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine tripropylamine, tromethamine and the like.

When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Particularly preferred are citric, hydrobromic, hydrochloric, maleic, phosphoric, sulfuric and tartaric acids.

The preparation of the pharmaceutically acceptable salts described above and other typical pharmaceutically acceptable salts is more fully described by Berg et al., “Pharmaceutical Salts,” J. Pharm. Sci., 1977:66:1-19.

As used herein, the term “composition” is intended to encompass a product comprising the specified ingredients in the specific amounts, as well as any product which results, directly or indirectly, from combination of the specific ingredients in the specified amounts.

When a compound according to this invention is administered into a human subject, the daily dosage will normally be determined by the prescribing physician with the dosage generally varying according to the age, weight, sex and response of the individual patient, as well as the severity of the patient's symptoms.

The maxi-K channel blockers used can be administered in a therapeutically effective amount intravaneously, subcutaneously, topically, transdermally, parenterally or any other method known to those skilled in the art. Ophthalmic pharmaceutical compositions are preferably adapted for topical administration to the eye in the form of solutions, suspensions, ointments, creams or as a solid insert. Ophthalmic formulations of this compound may contain from 0.01 to 5% and especially 0.5 to 2% of medicament. Higher dosages as, for example, about 10% or lower dosages can be employed provided the dose is effective in reducing intraocular pressure, treating glaucoma, increasing blood flow velocity or oxygen tension. For a single dose, from between 0.001 to 5.0 mg, preferably 0.005 to 2.0 mg, and especially 0.005 to 1.0 mg of the compound can be applied to the human eye.

The pharmaceutical preparation which contains the compound may be conveniently admixed with a non-toxic pharmaceutical organic carrier, or with a non-toxic pharmaceutical inorganic carrier. Typical of pharmaceutically acceptable carriers are, for example, water, mixtures of water and water-miscible solvents such as lower alkanols or aralkanols, vegetable oils, polyalkylene glycols, petroleum based jelly, ethyl cellulose, ethyl oleate, carboxymethylcellulose, polyvinylpyrrolidone, isopropyl myristate and other conventionally employed acceptable carriers. The pharmaceutical preparation may also contain non-toxic auxiliary substances such as emulsifying, preserving, wetting agents, bodying agents and the like, as for example, polyethylene glycols 200, 300, 400 and 600, carbowaxes 1,000, 1,500, 4,000, 6,000 and 10,000, antibacterial components such as quaternary ammonium compounds, phenylmercuric salts known to have cold sterilizing properties and which are non-injurious in use, thimerosal, methyl and propyl paraben, benzyl alcohol, phenyl ethanol, buffering ingredients such as sodium borate, sodium acetates, gluconate buffers, and other conventional ingredients such as sorbitan monolaurate, triethanolamine, oleate, polyoxyethylene sorbitan monopalmitylate, dioctyl sodium sulfosuccinate, monothioglycerol, thiosorbitol, ethylenediamine tetracetic acid, and the like. Additionally, suitable ophthalmic vehicles can be used as carrier media for the present purpose including conventional phosphate buffer vehicle systems, isotonic boric acid vehicles, isotonic sodium chloride vehicles, isotonic sodium borate vehicles and the like. The pharmaceutical preparation may also be in the form of a microparticle formulation. The pharmaceutical preparation may also be in the form of a solid insert. For example, one may use a solid water soluble polymer as the carrier for the medicament. The polymer used to form the insert may be any water soluble. non-toxic polymer, for example, cellulose derivatives such as methylcellulose, sodium carboxymethyl cellulose, (hydroxyloweralkyl cellulose), hydroxyethyl cellulose, hydroxypropyl cellulose, hydroxypropylmethyl cellulose; acrylates such as polyacrylic acid salts, ethylacrylates, polyactylamides; natural products such as gelatin, alginates, pectins, tragacanth, karaya, chondrus, agar, acacia; the starch derivatives such as starch acetate, hydroxymethyl starch ethers, hydroxypropyl starch, as well as other synthetic derivatives such as polyvinyl alcohol, polyvinyl pyrrolidone, polyvinyl methyl ether, polyethylene oxide, neutralized carbopol and xanthan gum, gellan gum, and mixtures of said polymer.

Suitable subjects for the administration of the formulation of the present invention include primates, man and other animals, particularly man and domesticated animals such as cats and dogs.

The pharmaceutical preparation may contain non-toxic auxiliary substances such as antibacterial components which are non-injurious in use, for example, thimerosal, benzalkonium chloride, methyl and propyl paraben, benzyldodecinium bromide, benzyl alcohol, or phenylethanol; buffering ingredients such as sodium chloride, sodium borate, sodium acetate, sodium citrate, or gluconate buffers; and other conventional ingredients such as sorbitan monolaurate, triethanolamine, polyoxyethylene sorbitan monopalmitylate, ethylenediamine tetraacetic acid, and the like.

The ophthalmic solution or suspension may be administered as often as necessary to maintain an acceptable IOP level in the eye. It is contemplated that administration to the mamalian eye will be about once or twice daily.

For topical ocular administration the novel formulations of this invention may take the form of solutions, gels, ointments, suspensions or solid inserts, formulated so that a unit dosage comprises a therapeutically effective amount of the active component or some multiple thereof in the case of a combination therapy.

The following examples given by way of illustration is demonstrative of the present invention.

### PREPARATIVE EXAMPLE 1

### Synthesis of 6-OMe-Indole

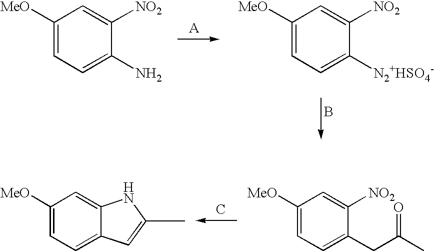 Step A

Adapted from ref: Magnus et al., J. Am. Chem. Soc. 110, 7, 2243, 1988.

4-Methoxy-2-nitro-aniline (35 g-Aldrich) was suspended in 250 mL of ethanol followed by addition of 14 mL of concentrated sulfuric acid. The suspension was cooled to 0° C., followed by slow addition of isoamyl nitrite (34 mL). After complete addition of isoamyl nitrite, the reaction mixture was stirred at 0 C for 1.5 h at which point a thick white slurry resulted. The reaction mixture was filtered and the precipitate was washed with 200 mL of cold ethanol followed by washing with 500 mL of ether. The filter cake was sucked dry under reduced pressure. 52 g of a free flowing powder was collected and used in the next step directly.

Step B

A 1 L flask was charged with isopeopenyl acetate (75 mL), acetone (400 mL), 0.25 M HCl (250 mL), Cu (II)Cl₂ (4 g) and LiCl (15 g). This was cooled to 0 C followed by portionwise addition of the diazonium salt obtained above. The reaction mixture was vented throughout the 18 h reaction time. The reaction mixture was concentrated to a viscous oil, diluted with ethyl acetate (200 mL) and washed with water (50 mL). The organic phase was collected, dried and concentrated to an orange-reddish oil which subjected to purification by SGC to provide colorless low melting product (16 g) LCMS=[M+H] 209

Step C

Compound obtained in step B was taken up in 200 mL of ethyl acetate followed by addition of 20 g of Raney Nickel (previously washed with ethyl acetate). The reaction mixture was subjected to reduction with hydrogen at atmospheric pressure for 12 h. After TLC analysis indicated complete conversion, the reaction mixture was filtered over a pad of celite and this was washed thoroughly with ethyl acetate and methanol. The combined organic extracts were concentrated to provide crystalline white product (12 g). LCMS: [M+H] 162. 1H NMR (CDCL, 500 MHz)): 7.8 (bs, 1H); 7.4 (d, 1H, J=XHz); 6.3-6.1 (m, 3H); 3.85 (s, 3H); 2.4 (s, 3H).

The compounds of this invention can be made, with modification where appropriate, in accordance with Schemes A and/or B. Examples 1-8 are also produced in accordance with Schemes A and/or B. 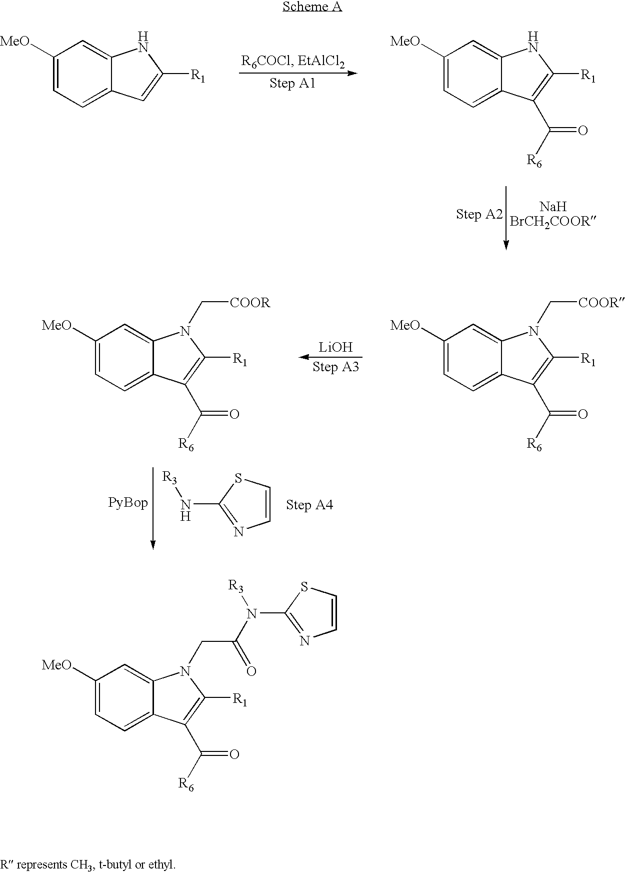
Step A1

6-methoxy indole (1 g, 6.2 mmole —Biochemica & Synthetica (Switzerland)) was charged into a 100 mL flask. After evacuation and purging with argon, 15 mL of dichloromethane was added followed by addition of EtAlCl₂ (9.92 mmoles, 5.5 mL of a 1.8M solution in toluene), the reaction mixture was allowed to stir for 15 min after which methyl magnesium chloride (6.2 mmole, 2mL of a 3M solution in ether) was added. This was allowed to stir for another 15 min when the reaction appeared cloudy. The requisite acid chloride (6.5 mmole) was added slowly and the reaction mixture was allowed to stir for an additional 0.5 h. The reaction mixture was diluted with 100 mL of ethyl acetate and quenched by addition of 20 mL of a saturated solution of ammonium chloride. The organic phases were separated and aqueous phase was back extracted with 50 mL portions of ethyl acetate twice. Combined organic extracts were dried over sodium sulphate and concentrated. The residue; which was usually colored was triturated with 20% ether in hexanes, filtered and dried to yield acylated product in all cases. This material was used in the Step B directly.

Step A2

4.8 mmoles of material obtained above was dissolved in 5 mL of dry DMF(dimethylformamide) followed by addition of 1 equiv. of NaH (sodium hydride). After evolution of all hydrogen gases had ceased, which usually took about 20 min, methyl bromo acetate (1.5 equiv.) was added portionwise. The reaction mixture was allowed to stir for another 1 h after which TLC (thin layer chromatography) analysis indicated complete consumption of all starting material. The reaction mixture was diluted with 50 mL of ethyl acetate and washed with brine (15 mL×2). The organic phase was dried over sodium sulphate and concentrated to yield an oil which was applied to a SG (silica gel) column and eluted with 20% ethly acetate in hexanes to yield alkylated product in all cases.

Step A3

Saponification of material obtained above was carried out using aqueous lithium hydroxide. 2.88 mmoles of ester was dissolved in 30 mL of THF (teterahydrofuran) and 2 quiv of LiOH (lithium hydroxide) was added as a 1M solution in water. The reaction mixture was stirred vigorously for 1 h. TLC analysis indicated complete reaction. The reaction mixture was then evaporated to half its original volume and diluted with 50 mL of ethyl acetate. 1M HCl solution was then added to bring the pH of the aqueous phase up to 2. The organic phase was separated and the aqueous phase was back extracted with 15 mL of ethyl acetate twice. Combined organic phases were dried over sodium sulphate and concentrated to a solid. This was azeotroped thoroughly by repeated evaporations with toluene in order to obtain a dry solid (about 2.7 mmoles in all cases), which was ready to be used for amide formation reactions.

Step A4

Amide formation was achieved using the peptide coupling reagent PyBOP ([bromo-tris-pyrrolidino-phosphonium hexafluorophosphate]-Novabiochem) as follows. Typically 0.3 mmole of starting acid was charged into a 100 mL flask, followed by the addition of PyBoP (0.6 mmoles) and the requisite amino thiazole (1.2 equiv., 0.36 mmole) under argon. The solvent acetonitrile (2 mL) was added followed by the addition of Hunigs base (0.9 mmoles). The reaction was sealed and heated to 100° C. for about 1 h at which time TLC analysis indicated complete reaction. The reaction mixture was evaporated and re-dissolved in 15 mL of ethyl acetate. This was passed through a small plug of silica gel and washed down with an additional 20 mL of ethyl acetate. The combined organic-phase was washed with brine, separated, dried over sodium sulphate and concentrated. The residue was purified by normal phase or reverse phase column chromatography. 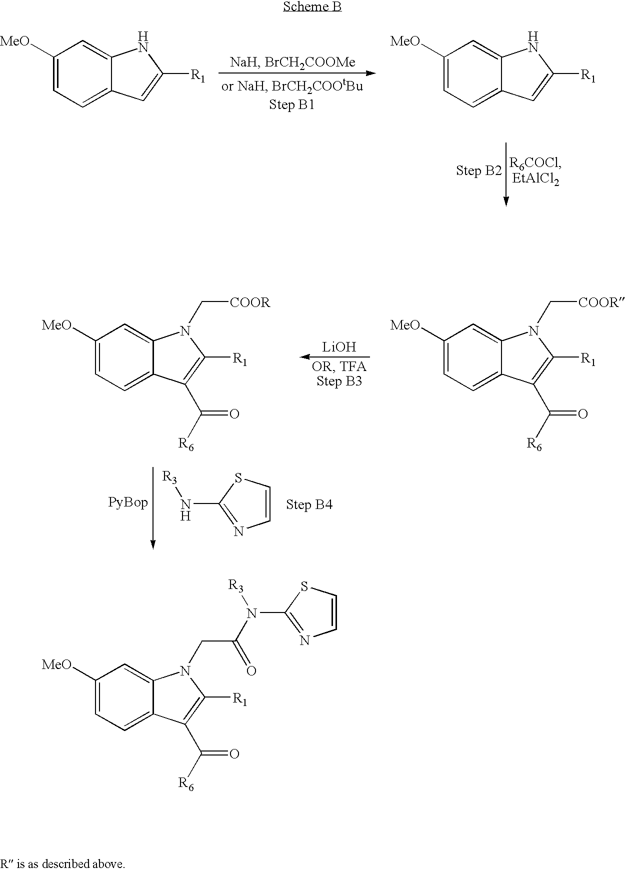
Step B1

1 g of 6-methoxy indole (Biochemica & Synthetica (Switzerland)) was dissolved in 10 mL of DMF (dimethylformamide) followed by the addition of 1.5 equiv of NaH (9.3 mmoles, 372 mg of a 60% solution in mineral oil). The reaction mixture was allowed to stir for 1 h at rt followed by the addition of 2 equiv of methyl bromo acetate or t-butyl bromo acetate. After 1 h the reaction was complete by TLC and it was subjected to standard aqueous work up. Purification of crude by SGC (silica gel chromatography) provided about 4.0-4.5 mmoles of product.

Step B2

4.0 mmoles of product obtained above was dissolved in 15 mL of dichloromethane under argon. The reaction mixture was cooled to 0° C. followed by the addition of 1.2 equiv of ethyl aluminum dichloride. After 1 h at 0° C. the requisite acid chloride (1.2 equiv) was added and the reaction mixture was stirred for an additional 1 h at 0° C. TLC (thin layer chromatography) analysis at this stage indicated complete reaction and the reaction was subjected to a standard aqueous work up. SGC (silica gel chromatography) purification provided about 3.5 mmoles of product.

Step B3a (hydrolysis of methyl ester)

The methyl ester obtained above (3.5 mmoles) was dissolved in 25 mL of THF (tetrahydrofuran) followed by the addition of 1.5 equiv of a 1M solution of LiOH in water. The reaction was stirred for 0.5 h at which time TLC analysis indicated complete hydrolysis. The reaction mixture was diluted with 15 mL of ethyl acetate and acidified with 2 mL of 1M HCl. The organic extracts were separated, dried over sodium sulfate and concentrated. The residue was suspended in toluene and evaporated twice to give the acid as white solid (3.3 mmoles) which was used in the next step directly.

Step B3b(hydrolysis of t-butyl ester)

The t-butyl ester obtained above (3.5 mmoles) was dissolved in 10 mL of dichloromethane followed by the addition of 5 mL of TFA (trifluoroaceticacid). The reaction was allowed to stir at rt for 1 h at which point TLC analysis indicated complete reaction. The solvent was stripped and resulting residue was resuspended in toluene and evaporated to dryness twice to the acid as a white solid (3.3 mmoles) which was used in the next step directly.

Step B4

Amide formation was achieved using the peptide coupling reagent PyBoP (Novabiochem) as follows. Typically 0.3 mmole of starting acid was charged into a 100 mL flask, followed by the addition of PyBoP (0.6 mmoles) and the requisite amino thiazole (1.2 equiv., 0.36 mmole) under argon. The solvent acetonitrile (2 mL) was added followed by the addition of Hunigs base (0.9 mmoles). The reaction was sealed and heated to 100° C. for about 1 h at which time TLC analysis indicated complete reaction. The reaction mixture was evaporated and redissolved in 15 mL of ethyl acetate. This was passed through a small plug of silica gel and washed down with an additional 20 mL of ethyl acetate. The combined organic phase was washed with brine, separated, dried over sodium sulphate and concentrated. The residue was purified by normal phase or reverse phase column chromatography.

### EXAMPLE 1

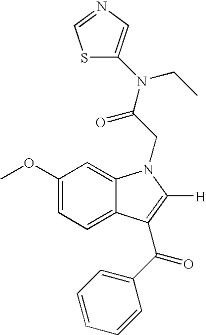

1H NMR (CDCl₃): 8.35 (1H, d, J=9 Hz); 7.85 (2H, bd, J=7.5 Hz); 7.6-7.48 (6H, m); 7.02 ( 1H, dd, J=9 Hz & 2 Hz); 5.2 (2H, bs); 4.4 (2H, bm); 3.9 (3H, s); 1.5 (3H, m). LCMS: [M+H]=420.

### EXAMPLE 2

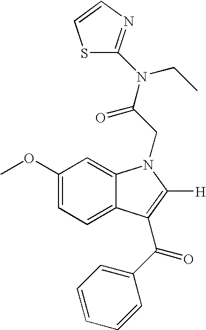

1H NMR (CDCl₃): 8.35 (1H, d, J=9 Hz); 7.85 (2H, bd, J=7.5 Hz); 7.6-7.48 (6H, m); 7.02 (1H, dd, J=9 Hz & 2 Hz); 5.2 (2H, bs); 4.4 (2H, bm); 3.9 (3H, s); 1.5 (3H, m). LCMS: [M+H]=420.

### EXAMPLE 3

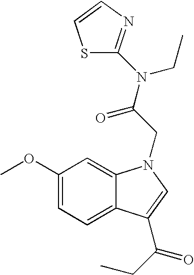

1H NMR (CDCl₃): 7.90 (1H, d, J=9 Hz); 7.60 (1H, d, J=3.5 H-z); 7.1 (1H, bs); 6.93 (1H, dd, J=9Hz & 2 Hz); 6.67 (1H, d, J=2 Hz); 5.2 (2H, bs); 4.4 (2H, bm); 3.9 (3H, s); 3.1 (2H, q); 1.5 (3H, m); 1.3 (3H, t). LCMS: [M+H]=372.

### EXAMPLE 4

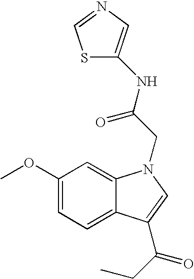

1H NMR (CDCl₃): 7.80(1H, d, J=9Hz); 7.41 (1H, d, J=4 Hz); 7.03 (1H, d, J=4 Hz); 6.84 (1H, dd, J=9 Hz & 2 Hz); 6.75 (1H, d, J=2 Hz); 5.1 (2H, s); 3.8 (3H, s); 3.0 (2H, q); 1.3 (3H, t). LCMS: [M+H]=344.

### EXAMPLE 5

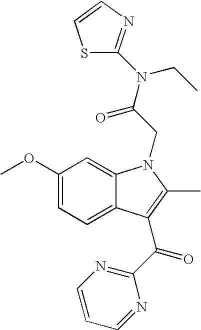

1H NMR (CDCl₃): 8.98 (2H, d, J=4.5 Hz); 7.63 (1H, d, J=3.5 Hz); 7.51 (2H, dd, J=5 Hz); ); 7.21 (1H, bs); 7.15 (1H, d, J=9 Hz); 6.79 (1H, dd, J=9 Hz & 2 Hz); 6.65 (1H, d, J=2 Hz); 5.2 (2H, bs); 4.4 (2H, bm); 3.9 (3H, s); 2.45 (3H, bs); 1.5 (3H, m). LCMS: [M+H]=436.

### EXAMPLE 6

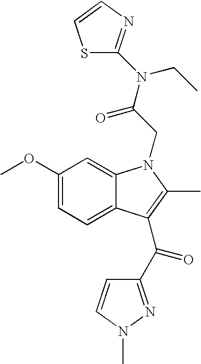

1H NMR (CDCl₃): 7.65 (1H, d, J=9 Hz); 7.63 (1H, d, J=3.5 Hz); 7.45 (1H, bs); 7.27 ( 1H, bs); 7.15 (1H, bs); 6.84 (1H, m); 6.62 (1H, bs); 5.2 (2H, bs); 4.4 (2H, bm); 3.9 (3H, s); 3.8 (3H, s); 2.45 (3H, bs); 1.5 (3H, m). LCMS: [M+H]=438.

### EXAMPLE 7

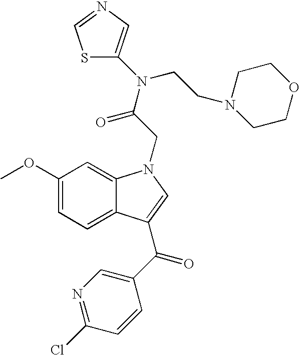

1H NMR (CDCl₃): 8.79 (1H, d, J=9 Hz); 7.85 (2H, bd, J=7.5 Hz); 7.6-7.48 (6H, m); 7.02 (1H, dd, J=9 Hz & 2 Hz); 5.2 (2H, bs); 4.4 (2H, bm); 3.9 (3H,s); 1.5 (3H, m). LCMS: M+H]=420.

### EXAMPLE 8

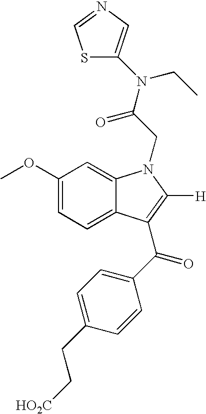

Mass spectrum (ESI) 492 (M+1). 1H NMR (500 MHz, DMSO-d₆): δ1.42 (t, 3H, J=6.8 Hz); 2.56(t, 2H, J=7.5 Hz); 2.90(t, 2H, J=7.5 Hz); 3.78(s, 3H); 4.32(m, 2H); 5.63(s, 2H); 6.91(dd, 1H, J=8.5, 2.0 Hz); 7.17(d, 1H, J=1.5 Hz); 7.28(d, 1H, J=3.0 Hz); 7.39(d, 2H, J=8.0 Hz); 7.56(d, 1H, J=3.5 Hz); 7.69(d, 2H, J=8.0 Hz); 8.01(s, 1H); 8.14(d, 1H, J=8.5 Hz).

### EXAMPLE 9

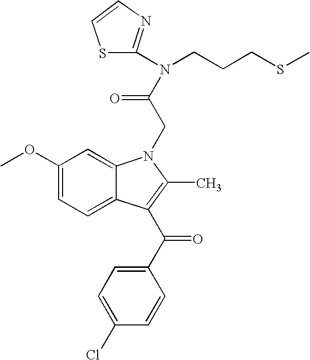

1H NMR (CDCl₃): 7.77 (2H, d); 7.58 (1H, bs); 7.45 (2H, d); 7.25 (1H, d,); 7.10 (1H, bd), 6.77 (1H, dd); 6.67 (1H, d); 5.30 (2H, bs); 4.48 (2H, bm); 3.84 (3H, s); 2.70 (2H, bm); 2.50 (3H, s); 2.28 (2H, bm); 2.21 (3H, bs).

The compounds illustrated in Examples 10-12 were prepared as shown in Schemes A and B above but substituting the appropriately substituted amine in either Step A4 or B4 for the substituted amino thiazole shown in the schemes.

### EXAMPLE 10

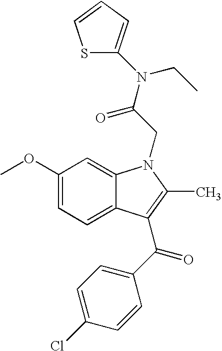

1H NMR (CDCl₃): 7.72 (2H, d); 7.42 (2H, d); 7.39 (1H, dd); 7.20 (1H, d,); 7.08 (1H, dd), 7.04 (1H, dd); 6.74 (1H, dd); 6.62 (1H, d); 4.72 (2H, s); 3.87 (3H, s); 3.84 (2H, q); 2.45 (3H, s); 1.22 (3H, t). LCMS (M+H)=467.

### EXAMPLE 11

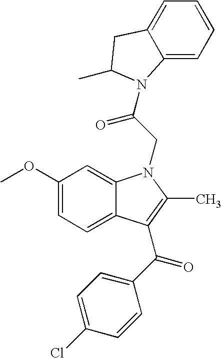

1H NMR (DMSO): 7.88(1H, d); 7.64 (2H, d); 7.59 (2H, d,); 7.32 (1H, d), 7.10 (4H, m); 6.74 (1H, dd); 5.58 (1H, d); 5.24 (1H, d); 4.90 (1H, m); 3.76 (3H, s); 3.50 (1H, m); 2.78 (1H, d); 2.49 (3H, s); 1.40 (3H, d). LCMS (M+H)=473.

### EXAMPLE 12

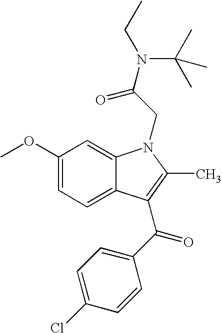

1H NMR (CDCl₃): 7.76 (2H, d); 7.44 (2H, d); 7.19 (1H, d); 6.75 (1H, dd); 6.65 (1H, d); 4.86 (2H, s); 3.85 (3H, s); 3.55 (2H, q); 2.51 (3H, s); 1.50 (9H, s); 1.43 (9H, s). LCMS (M+H)=441.

### EXAMPLE 13

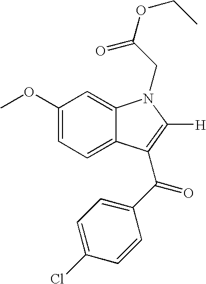

1H NMR (DMSO): 8.10 (1H, d); 7.94 (1H, s); 7.76 (2H, d); 7.60 (2H, d,); 7.14 (1H, d), 6.93 (1H, dd); 5.20 (2H, s); 4.17 (2H, q); 3.80 (3H, s); 1.22 (3H, t). LCMS ((M+H)=372.

### EXAMPLE 14

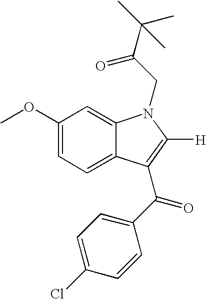

1H NMR (CDCl₃): 7.73 (2H, d); 7.44 (2H, d); 7.20 (1H, d); 6.74 (1H, d); 6.51 (1H, s); 5.01 (2H, s); 3.82 (3H, s); 2.37 (3H, s); 1.37 (9H, s). LCMS (M+H)=398. 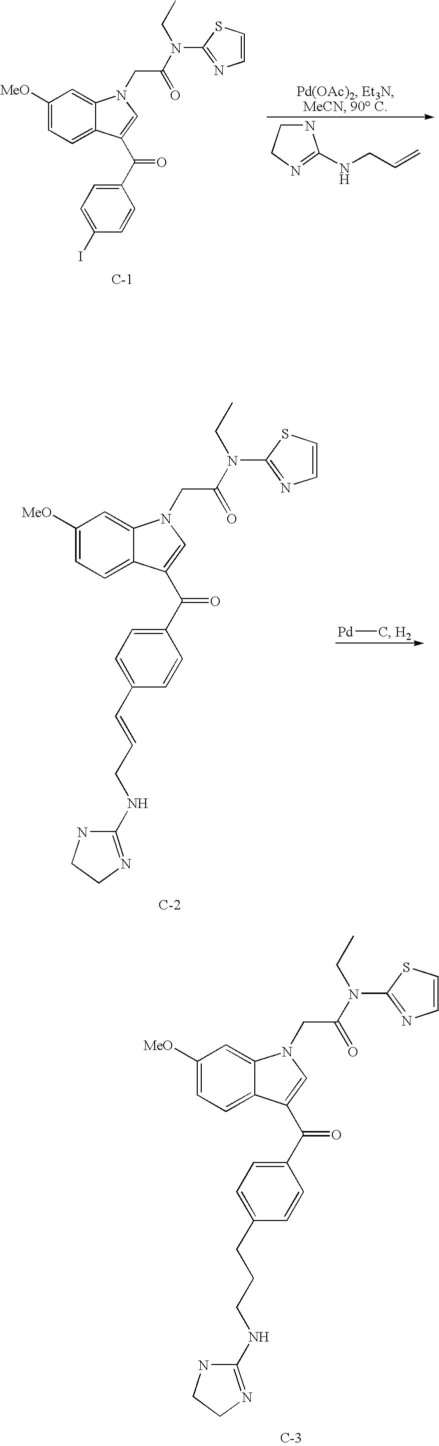
Step 1 Allyl amino-imidazoline

2-Methyl thio-2-imidazoline hydroiodide (1 mmole) was mixed with allyl amine (2 mmole) in 10 mL of dichloromethane at room temperature. The reaction was stirred for 12 h at which point TLC analysis indicated completion of reaction. Reaction mixture was concentrated and the residual oil was applied to SGC and eluted with 1-5% methanol in dichloromethane. 0.9 mmole of desired product was obtained as an oil.

1H NMR (DMSO-d₆): 8.39 (1H, bm); 5.8 (1H, m); 5.2 (2H, m); 3.8 (2H, bm); 3.6 (4H, bs);

Step 2

Compound C-1 (1 g, 1.83 mmoles, prepared as shown in Scheme A) was charged into a 100 mL flask followed by the addition of Pd(OAc)₂ (10 mol %). 15 mL of acetonirile was added followed by the addition of triethyl amine (2.5 equiv.) and allyl aminoimidazoline (1.2 equiv., 2.2 mmoles)¹. The reaction mixture was purged with argon, sealed and heated at 80° C. for 7 h at which time TLC analysis indicated the completion of reaction. The reaction mixture was filtered and concentrated to 20% original volume. This was loaded onto a reverse phase HPLC column and purified to provide instant compound C-2. Product obtained was used in the hydrogenation step. LCMS [M+H]=543

Step 3

The product obtained above was dissolved in methanol (10 mL) followed by addition of Pd—C (10%) and the reaction was evacuated and back purged with hydrogen using a balloon. TLC analysis indicated that reaction was complete after 1 h. The reaction mixture was filtered over a pad of celite. Concentration and purification reverse phase HPLC provided desired Compound C-2 as a white solid.

1HNMR: (DMSO-d₆): δ 8.10 (1H, d, J=8.5 Hz); 7.77 (3H, m); 7.53 (1H, bs); 7.37 (2H, d, J=8 Hz); 6.97 (1H, m); 6.94 (1H, m); 5.51 (2H, bs); 4.36 (1H, bs); 83.82 (3H, s); 3.69 (4H, bs); 3.2 (2H, m); 2.8 (2H, m); 1.9 (2H, m); 1.45 (2H, bs); LCMS [M+H]=545. 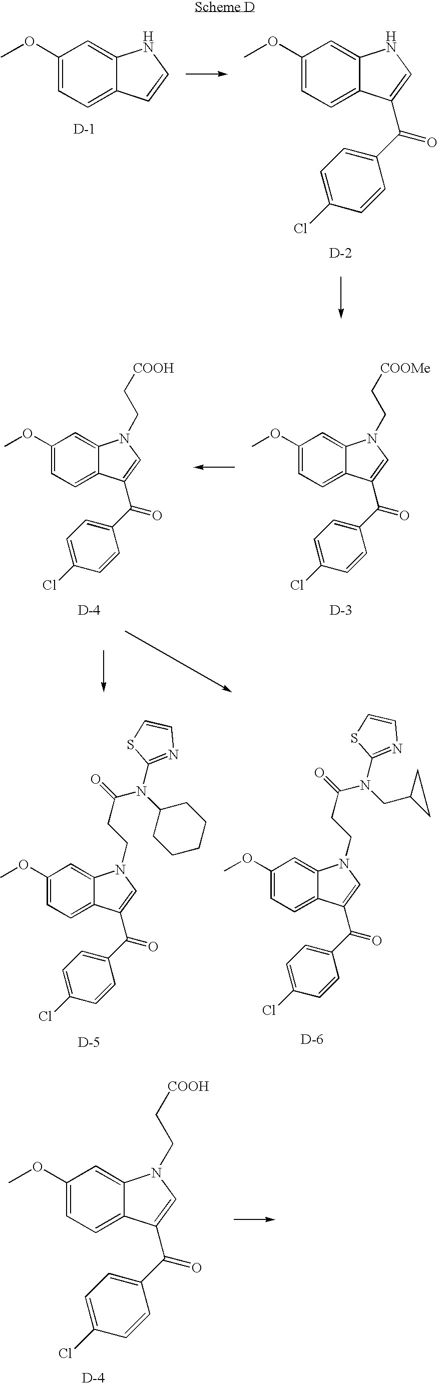 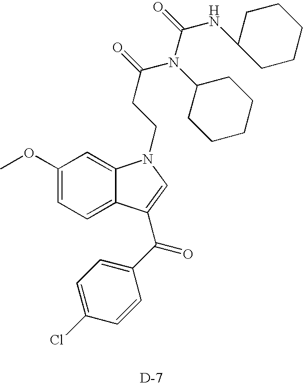
Procedure
Step 1

Compound D-2 was obtained as described in general scheme A above.

Step 2

Compound D-4 was synthesized as follows: 1.8 g of compound D-2 was dissolved in 6 mL of DMF followed by addition of sodium hydride (1.2 equiv.). The reaction was allowed to stir for 0.5 h at room temperature then methyl-3-bromopropionate was added (1.5 equiv.). The reaction was allowed to stir for 0.5 h at which point TLC analysis indicated complete consumption of starting material. The reaction was poured into 50 mL of water, stirred for 0.5 h at which time the resultant solids formed were collected by filtration and dried thoroughly. The resulting solids were dissolved in 30 mL of THF followed by the addition of a solution of 1M LiOH in water. The reaction was stirred for 1 h at which point TLC analysis indicated completion of reaction. The solvents were evaporated and upon acidification the resulting solids were collected and dried thoroughly before use in te next step.

Step 3

100 mg of acid D-4 obtained in the Step 2 was charged into a 100 mL flask followed by the addition of pyBop (2 equiv.) and cyclohexyl amino thiazole (1.2 equiv) and Hunigs base (3.5 equiv.). The solvent used was acetonitrile (10 mL). The reaction was heated in an inert atmosphere for 1 h. standard aqueous and purification provided 19% yield of desired compound D-5 [M+H]=522.

Compound D-6

Using a similar procedure (as described for the preparation of compound D-5) on a 100 mg scale, acid D-4 was coupled with cyclopropyl methyl amino thiazole to provide compound D-6. [M+H]=494.

Compound D-7

100 mg of acid D-4 was treated with 1.5 equiv. of dicylohexyl carbodiimide in 10 mL of dichloromethane. The reaction mixture was heated to reflux for 1 h after which the reaction was concentrated nd purified using silica gel chromatography to provide 69% of desired compound G [M+H]=564.

### EXAMPLE 15

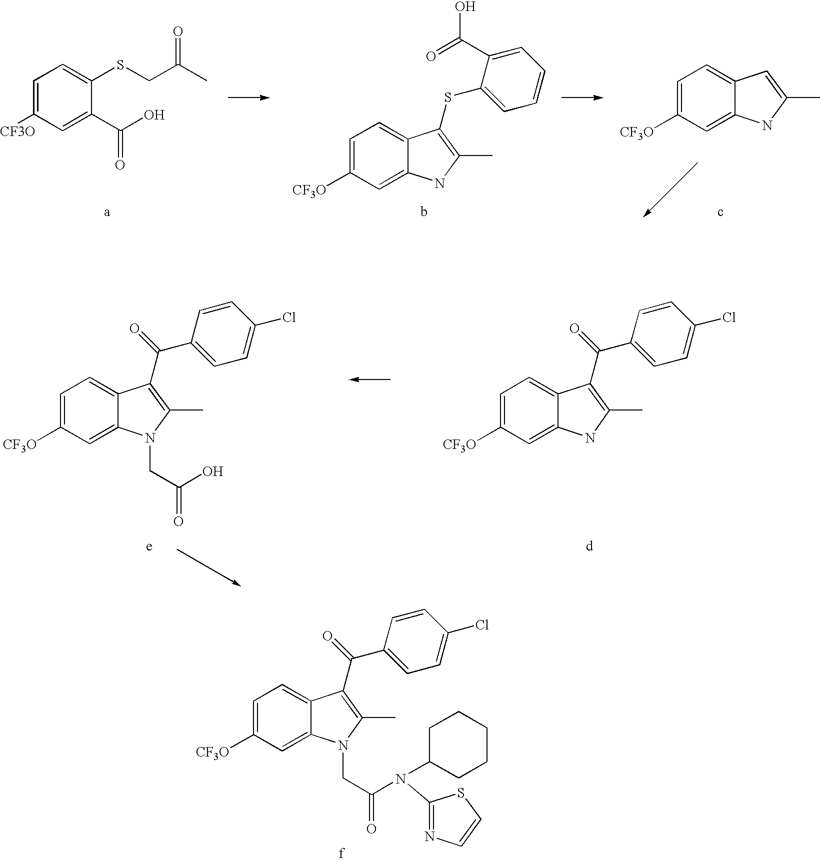

A suspension of chloroacetone (6.00 grams, 65 mmol, filtered through basic alumina prior to use), phenol 1 (10.00 grams, 65 mmol) and potassium carbonate (8.96 grams, 65 mmol) was stirred in DMF at room temperature under nitrogen atmosphere for 1 hour. The was then diluted with ethyl acetate/H₂O and the layers separated. The aqueous layer was acidified with 1N HCl and extracted with ethyl acetate (3×). The organic layer was then washed with water (2×), and brine (1×), dried with sodium sulfate, filtered and evaporated to give intermediate a;

¹H-NMR (CDCl₃ 500 MHz) δ 8.14 (t, 1H), 7.53 (t, 1H), 7.35 (d, 1H), 7.27 (d, 1H), 3.78 (s, 2H), 2.35 (s, 3H).

Intermediate a (1.84 grams, 8.75 mmol) and 4-trifluoromethoxy phenylhydrazine hydrochloride (2.00 grams, 4.76mmol) were stirred at 100° C. in acetic acid (40 mL, 0.22M) for 1 hour under nitrogen atmosphere to give a 1:2 mixture of 4- and 6-trifluoromethoxy indoles. The reaction was cooled to room temperature, the acetic acid was removed under reduced pressure and the residue was diluted with ethyl acetate and washed with water (1×) and brine (1×). The organic layer was dried with sodium sulfate, filtered and evaporated to afford intermediate compound b as a yellow oil after chromatography (hexanes/ethyl acetate/1% acetic acid, 6:1) ¹H-NMR (CDCl₃ 500 MHz) δ 8.43 (br s, 1H), 8.16 (dd, 1H), 7.46 (d, 1H), 7.23 (t, 1H), 7.14 (t, 1H), 7.03 (d, 1H), 6.74 (d, 1H), 2.54 (s, 3H).

A solution of ntermediate b (0.29 grams, 0.78 mmol) and thiosalicylic acid (0.12 grams, 0.78 mmol) in trifluoroacetic acid (3 mL, 0.26M) was heated to 50° C. under nitrogen atmosphere for 2 hours. After this time the reaction was cooled to room temperature, diluted with ethyl acetate and washed with 1N NaOH (2×), and brine (1×). The organic layer was dried with sodium sulfate, filtered and evaporated to afford compound c;

1H-NMR (CDCl₃ 500 MHz) δ 8.01 (br s, 1H), 7.49 (d, 1H), 7.17 (s, 1H), 6.99 (d, 1H), 6.26 (s, 1H), 2.46 (s, 3H).

Zinc Chloride (0.23 grams, 1.66 mmol) and ethyl magnesium bromide (0.29 mL of a 3M solution in ether, 0.87 mmol) were added to a solution of compound c (0.16 grams, 0.74 mmol) in CH2Cl2. The resulting mixture was stirred at room temperature under a nitrogen atmosphere for 1 hour. 4-chlorobenzoyl chloride (0.21 grams, 1.18 mmol) was then added and stirring was continued for 1 hour. Aluminum chloride (0.053 grams 0.39 mmol) was added and the reaction mixture was stirred for 3 hours. The reaction was then quenched with NH4Cl(aq), diluted with CH2Cl2, washed with 1N NaOH (1×) and brine (3×). The organic layer was dried with sodium sulfate, filtered and evaporated to afford compound d after chromatography (hexanes/ethyl acetate, 4:1); ¹H-NMR (CDCl₃ 500 MHz) δ 8.54 (br s, 1H), 7.73 (d, 2H), 7.48 (d, 2H), 7.40 (d, 1H), 7.24 (s, 1H), 7.02 (d, 1H), 2.60 (s, 3H).

A solution of compound d (101 milligrams, 0.286 mmol), methyl bromoacetate (51 milligrams, 0.342 mmol) and Cs2CO3 (121 milligrams, 0.342 mmol) was stirred in DMF (1.4 mL) at toom temperature for 18 hours. The reaction was diluted with ether then washed with water (3×), brine(1×), dried, filtered, and evaporated to afford a light yellow solid that was saponified without purification. The ester was stirred with NaOH (0.340 mL, 1.0 M aq.) in THF/MeOH (3:1) for 18 hours. The reaction was diluted with ether and acidified with 1N HCl to pH 3. The organic layer was separated and washed with water (2×), brine (1×) then dried filtered and evaporated to give compound e;

¹H-NMR (CDCl₃ 500 MHz) δ 7.74 (d, 8.6 Hz, 2H), 7.45 (d, 8.6 Hz, 2H), 7.33 (d, 8.7 Hz, 1H), 7.13 (br s, 1H), 7.04 (br d, 8.7 Hz), 4.92 (s, 2H), 2.53 (s, 3H).

Triethylamine (42 uL, 0.30 mmol), PyBrOP (70 mg, 0.15 mmole), and compound e (31 milligrams, 0.075 mmol) were added sequentially to a suspension of N-cyclohexyl-2-amino thiazole (14 milligrams, 0.075 mmol) in acetonitrile (200 uL). The clear brown solution was heated at 100 C for 1.5 hours. The reaction was cooled to room temperature and diluted with ethyl acetate. The ethyl acetate was washed with water (1×), and brine (1×) then dried filtered and evaporated to give a crude residue that was purified by C-18 HPLC (acetonitrile:water, 10:90-100:0, gradient elution over 15 minutes) to give compound f; ¹H-NMR (CDCl₃ 500 MHz) δ 7.83 (d, 6.3 Hz, 1H), 7.71 (d, 8.3 Hz, 2H), 7.52 (d, 3.7 Hz, 1H), 7.44 (d, 8.3 Hz, 2H), 7.31 (d, 8.7 Hz, 1H), 7.03 (br s, 1H), 6.97 (br d, 8.7 Hz, 1H), 4.55 (s, 2H), 4.53 (m, 1H), 2.46 (s, 3H), 1.93 (m, 2H), 1.81 (m, 2H), 1.64 (m, 1H), 1.37 (m, 4H), 1.03 (m, 1H), MS (M+1) 576.

### EXAMPLE-16

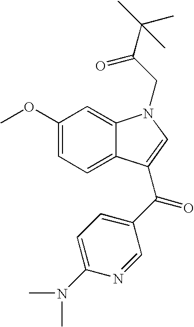

1H NMR (CDCl3): 8.77 (1H, d); 8.30 (1H, d); 8.11 (1H, dd); 7.49 (1H, s); 6.98 (1H, dd); 6.65 (1H, d); 6.57 (1H, d); 5.07 (2H, s); 3.88 (3H, s); 3.25 (6H, s); 1.35 (9H, s). LCMS (M+H)=394.3.

### EXAMPLE 17

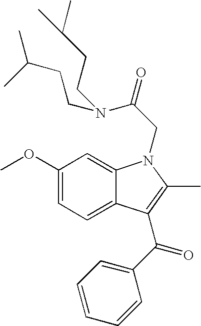

¹H NMR (CDCl₃) δ 0.942 (6 H, d), 1.044 (6 H, d), 1.483 (2 H, m), 1.613 (4 H, m), 1.683 (1 H, m), 2.449 (3 H, s), 3.395 (4 H, m), 3.840 (3 H, s), 4.817 (2 H, s), 6.605 (1H, s), 6.740 (1 H, d), 7.257 (1 H, d), 7.436 (2 H, m), 7.546 (1 H, m), 7.794 (2 H, m).

### EXAMPLE 18

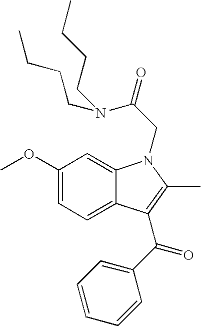

¹H NMR (CDCl₃) δ 0.956 (3 H, m), 1.047 (3 H, m), 1.340 (2 H, m), 1.467 (2 H, m), 1.578 (2 H, m), 1.704 (2 H, m), 2.487 (3 H, s), 3.399 (4 H, s), 3.837 (3 H, s), 4.869 (2 H, s), 6.642 (1 H, s), 6.747 (1 H, d), 7.227 (1 H, d), 7.472 (2 H, m), 7.565 (1 H, m), 7.801 (2 H, m).

### EXAMPLE 19

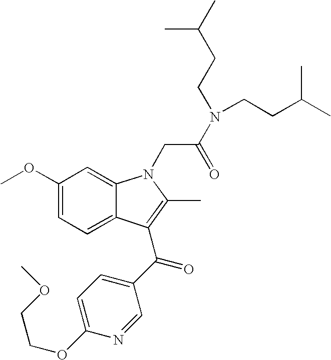

¹H NMR (CDCl₃) δ 0.930 (6 H, d), 1.035 (6 H, d), 1.484 (2 H, m), 1.612 (2 H, m), 1.713 (2 H, m), 2.507 (3 H, s), 3.413 (4 H, m), 3.482 (3 H, s), 3.811 (2 H, m), 3,853 (3 H, s), 4.592 (2 H, m), 4.850 (2 H, s), 6.636 (1 H, s), 6.765 (1 H, d), 6.860 (1 H, d), 7.303 (1 H, m), 8.037 (1 H, d), 8.614 (1 H, s).

### EXAMPLE 20

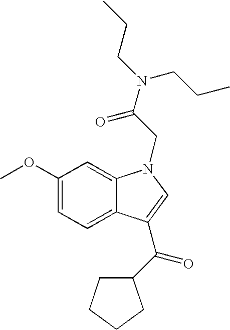

¹H NMR (CDCl₃) δ 0.928 (3 H, t), 0.987 (3 H, t), 1.641 (6 H, m), 1.794 (2 H, m), 1.916 (2 H, m), 1.989 (2 H, m), 3.315 (2 H, m), 3.358 (2 H, m), 3.518 (1 H, m), 3.871 (3 H, s), 4.888 (2 H, s), 6.693 (1 H, s), 6.947 (1 H, d), 7.734 (1 H, s), 8.322 (1 H, d).

### EXAMPLE 21

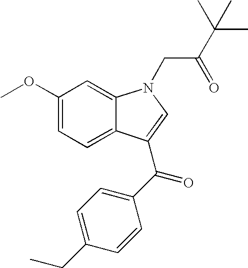

¹H NMR (CDCl₃) δ 1.321 (12 H, m), 2.738 (2 H, q), 3.855 (3 H, s), 5.002 (2 H, s), 6.537 (1 H, s), 6.991 (1 H, d), 7.301 (2 H, d), 7.398 (1 H, s), 7.792 (2 H, d), 8.351 (1 H, d).

### EXAMPLE 22

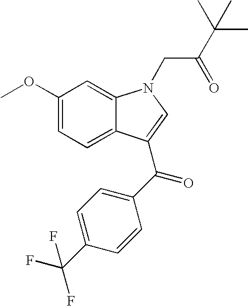

¹H NMR (CDCl₃) δ 1.345 (9 H, s), 3.876 (3 H, s), 5.049 (2 H, s), 6.573 (1 H, s), 7.007 (1 H, d), 7.361 (1 H, s), 7.764 (2 H, d), 7.938 (2 H, d), 8.353 (1 H, d).

### EXAMPLE 23

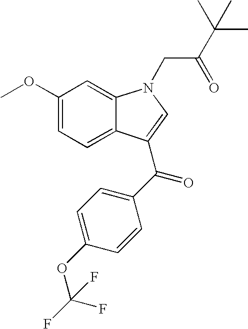

¹H NMR (CDCl₃) δ 1.351 (9 H, s), 3.883 (3 H, s), 5.068 (2 H, s), 6.587 (1 H, s), 7.022 (1 H, d), 7.344 (2 H, d), 7.401 (1 H, s), 7.911(2 H, d), 8.327 (1 H, d).

### EXAMPLE 24

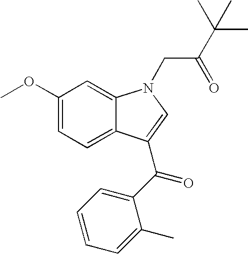

¹H NMR (CDCl₃) δ 1.324 (9 H, s), 2.421 (3 H, s), 3.863 (3 H, s), 4.992 (2 H, s), 6.557 (1 H, s), 7.008 (1 H, d), 7.180 (1 H, s), 7.294 (2 H, m), 7.366 (1 H, m), 7.459 (1 H, d), 8.323 (1 H, d).

### EXAMPLE 25

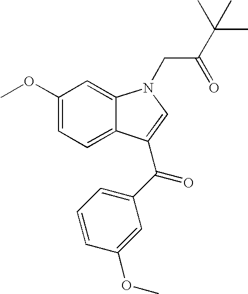

¹H NMR (CDCI₃) δ 1.292 (9 H, s), 3.771 (3 H, s), 3.833 (3 H, s), 4.911 (2 H, s), 6.457 (1H, s), 6.992 (3 H, m), 7.175 (1 H, s), 7.416 (2 H, m), 8.314 (1 H, d).

### EXAMPLE 26

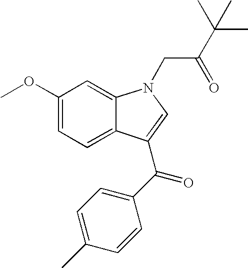

¹H NMR (CDCl₃) δ 1.328 (9 H, s), 2.455 (3 H, s), 3.867 (3 H, s), 5.043 (2 H, s), 6.574 (1 H, s), 6.987 (1 H, d), 7.286 (2 H, m), 7.415 (1 H, s), 7.782 (2 H, d), 8.326 (1 H, s).

### EXAMPLE 27

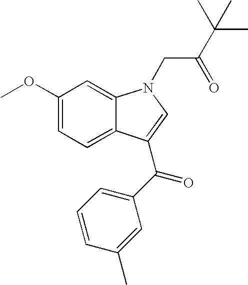

¹H NMR (CDCl₃) δ 1.283 (9 H, s), 2.449 (3 H, s), 3.870 (3 H, s), 5.033 (2 H, s), 6.558 (1 H, s), 6.988 (1 H, d), 7.368 (3 H, m), 7.656 (2 H, m), 8.335 (1 H, d).

### EXAMPLE 28

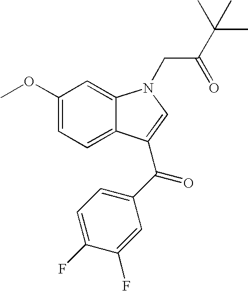

¹H NMR (CDCl₃) δ 1.358 (9 H, s), 3.879 (3 H, s), 5.077 (2 H, s), 6.580 (1 H, s), 7.020 (1 H, d), 7.294 (1 H, m), 7.405 (1 H, s), 7.650 (1 H, m), 7.701 (1 H, m), 8.292 (1 H, d).

### EXAMPLE 29

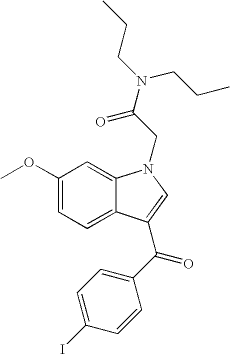

¹H NMR (CDCl₃) δ 0.909 (3 H, t), 1.012 (3 H, t), 1.610 (4 H, m), 3.328 (4 H, m), 3.902 (3 H, s), 4.893 (2 H, s), 6.721 (1 H, s), 6.996 (1 H, d), 7.482 (1 H, s), 7.590 (2 H, d), 7.853 (2 H, d), 8.318 (1 H, d).

### EXAMPLE 30

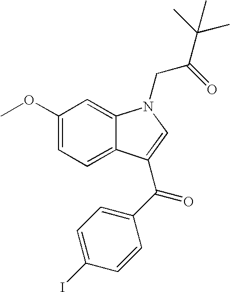

¹H NMR (CDCl₃) δ 1.333 (9 H, s), 3.881 (3 H, s), 5.057 (2 H, s), 6.578 (1 H, s), 7.014(1 H, d), 7.382 (1 H, s), 7.576 (2 H, d) 7.846 (2 H, d), 8.328 (1 H, d).

### EXAMPLE 31

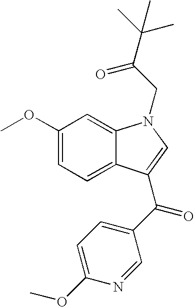

¹H NMR (CDCl₃) δ 1.259 (9 H, s), 3.848 (3 H, s), 4.066 (3 H, s), 5.091 (2 H, s), 6.553 (1 H, s), 6.967 (1 H, d), 6.984 (1 H, d), 7.521 (1 H, s), 8.187 (1 H, d), 8.273 (1 H, d), 8.724 (1 H, s).

### EXAMPLE 32

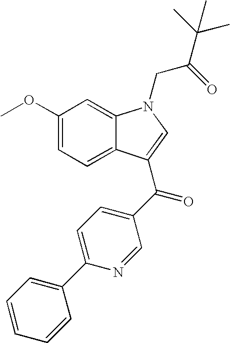

¹H NMR (CDCl₃) δ 1.356 (9 H, s), 3.893 (3 H, s), 5.088 (2 H, s), 6.600 (1 H, s), 7.029 (1 H, d), 7.506 (4 H, m), 7.901 (1 H, d), 8.100 (2 H, d), 8.271 (1 H, d), 8.376 (1 H, d), 9.173 (1 H, s).

### EXAMPLE 33

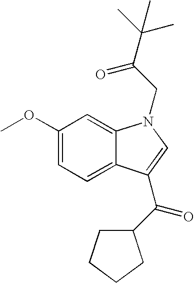

¹H NMR (CDCl₃) δ 1.353 (9 H, s), 1.647 (2 H, m), 1.796 (2 H, m), 1.926 (2 H, m), 2.000 (2 H, m), 3.509 (1 H, m), 3.851 (3 H, s), 5.036 (2 H, s), 6.530 (1 H, s), 6.945 (1 H, d), 7.617 (1 H, s), 8.342 (1 H, d).

### EXAMPLE 34

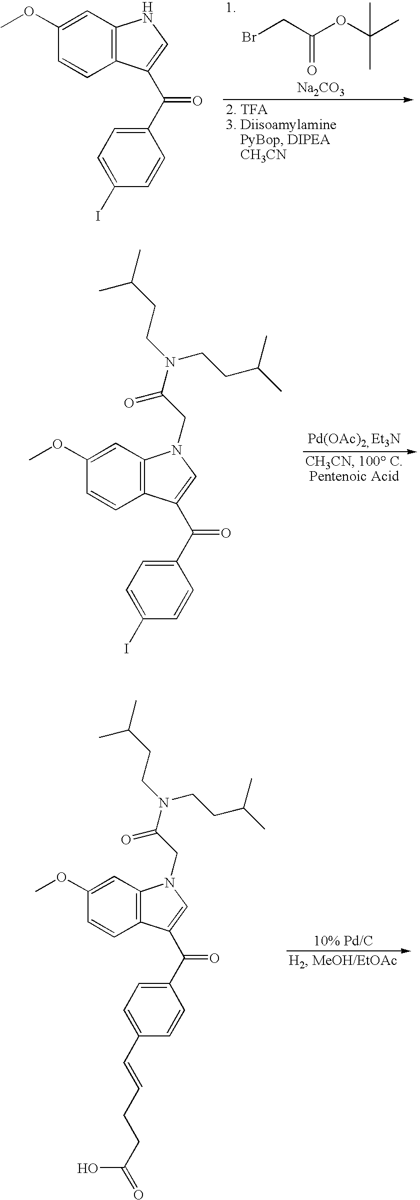

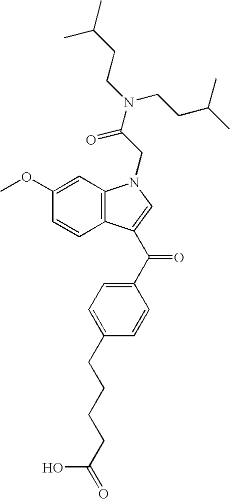

### EXAMPLE 35

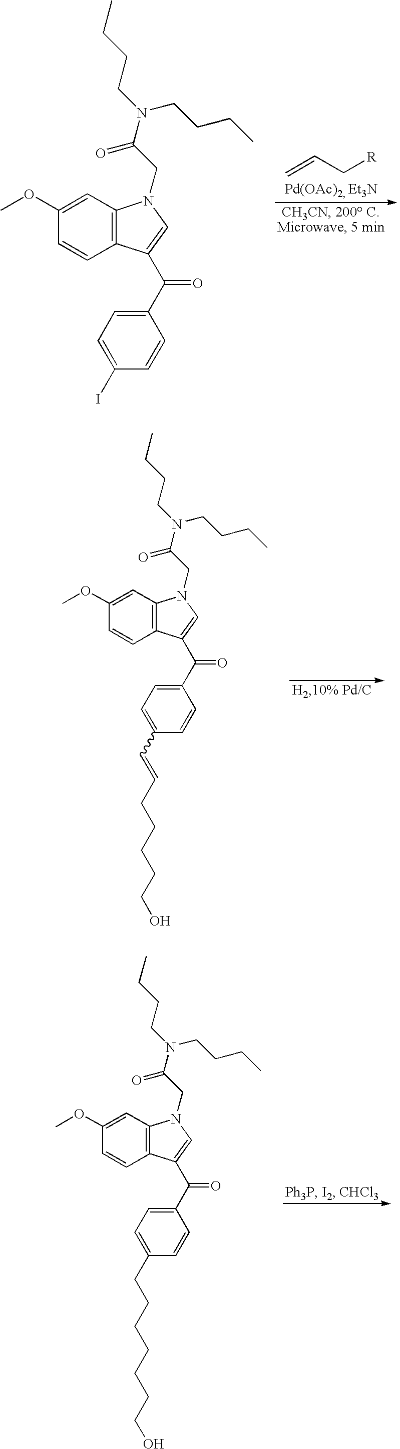

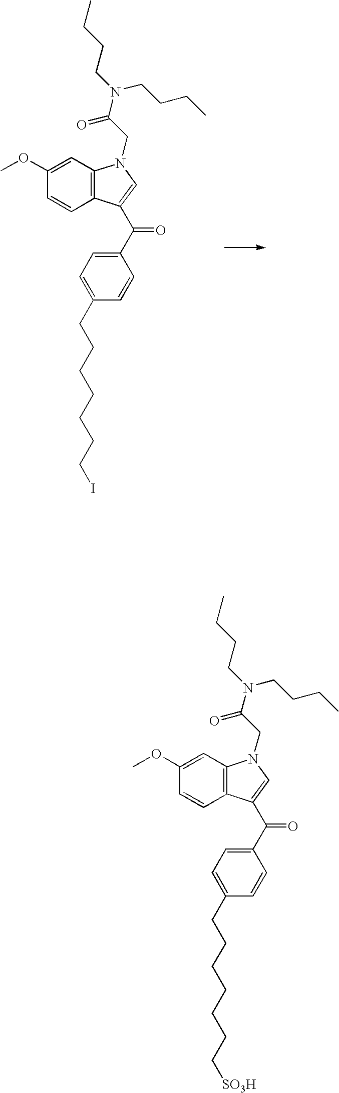

### EXAMPLE 36

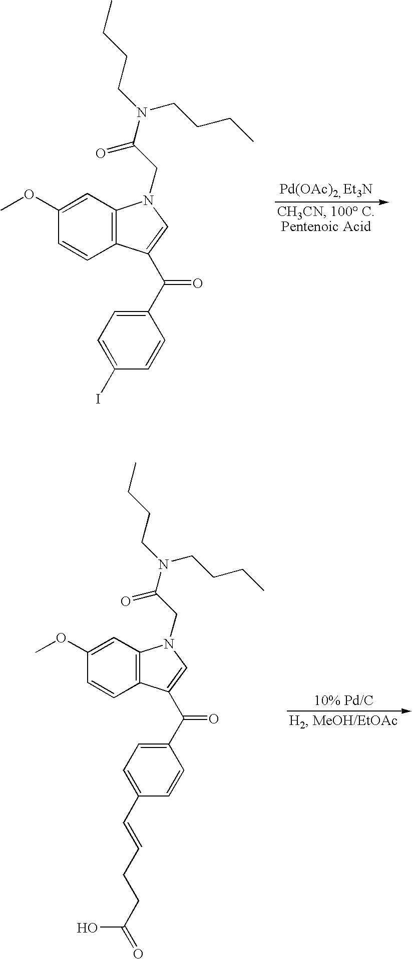

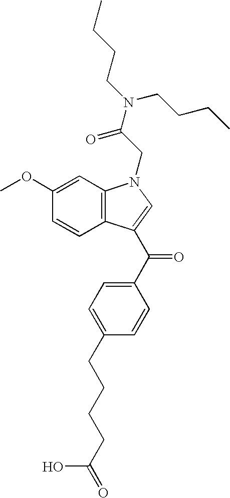

### EXAMPLE 37

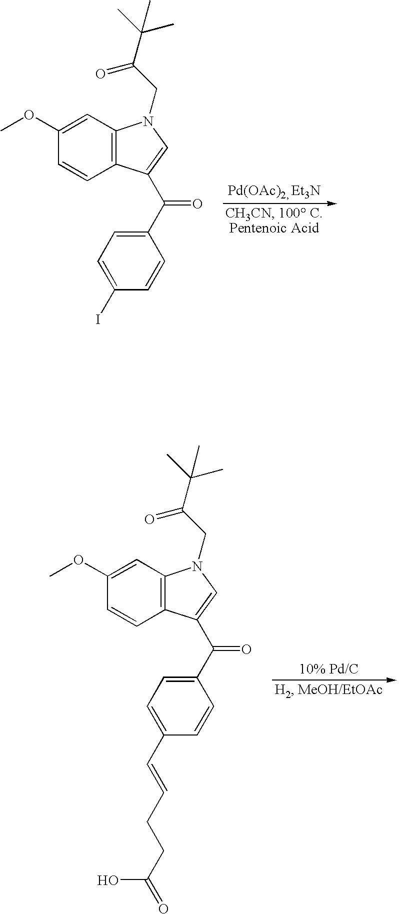 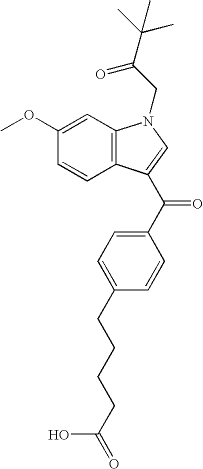 The compounds below are made by modifying Example 35 in a manner known to those skilled in the art.

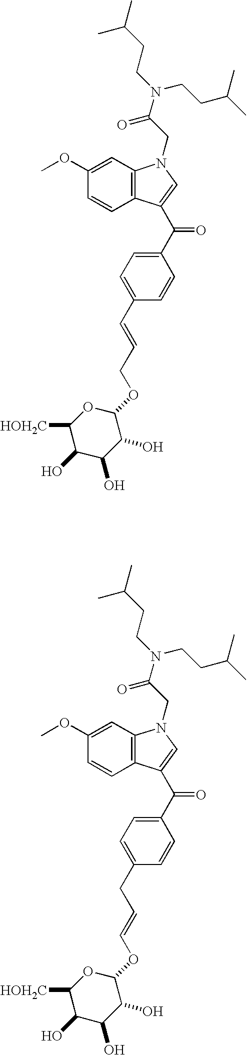

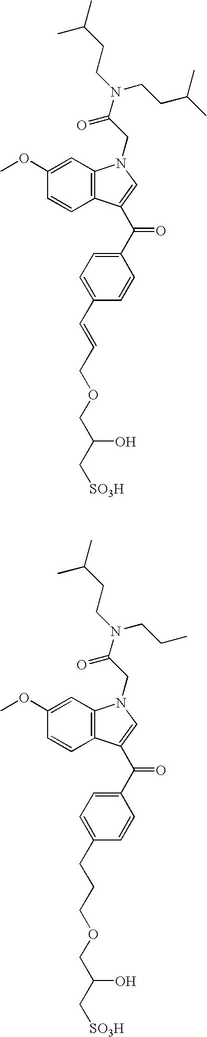

Using schemes E, F and G below, the compounds in the tables 7-10 were prepared:

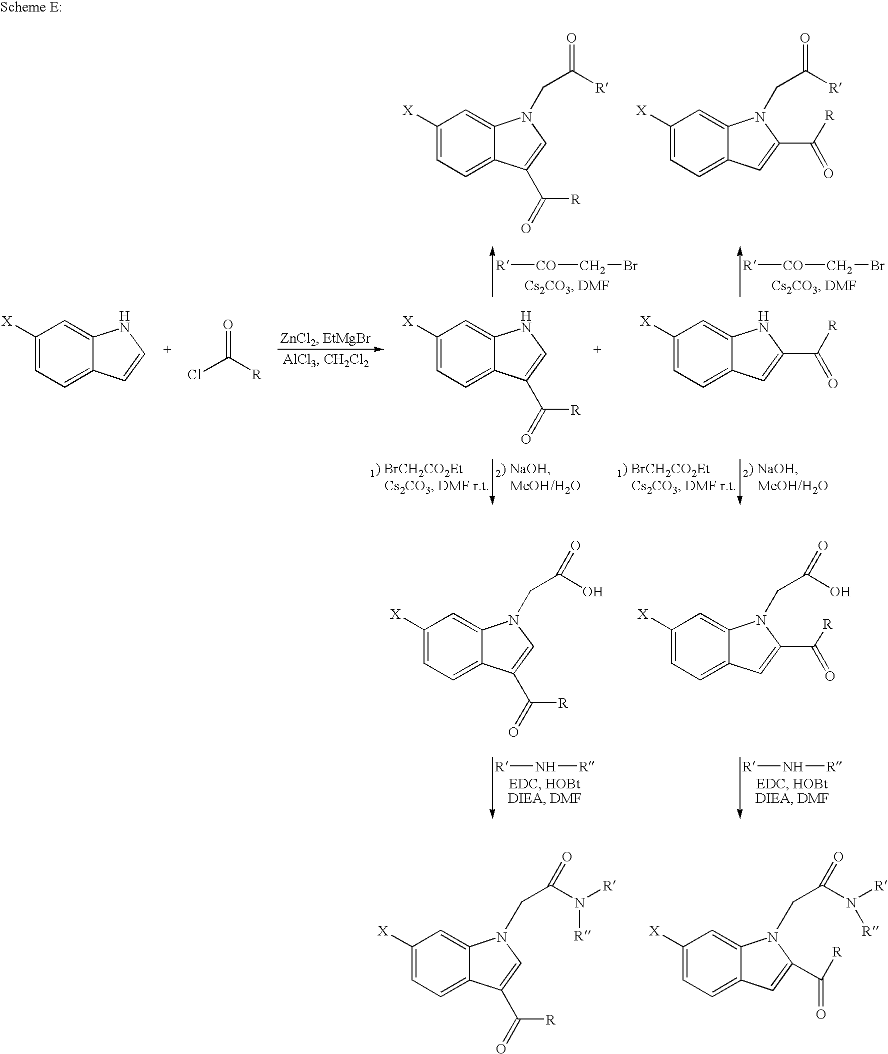

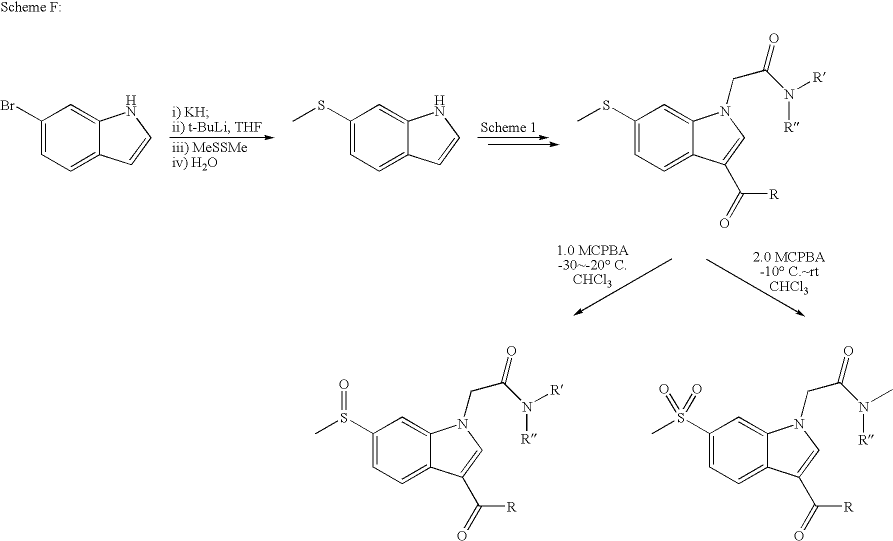

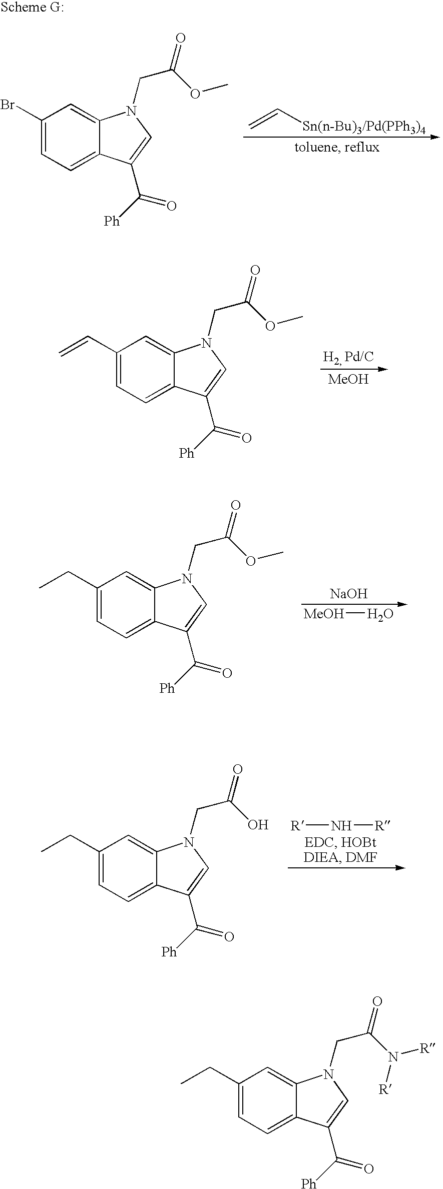

TABLE 7

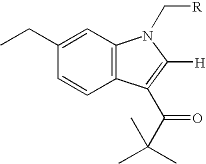

Y = O, or S(O)v, and v = 0-2

R is:

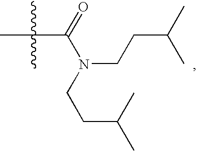

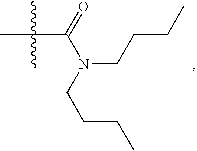

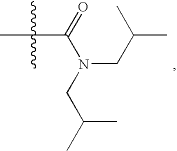

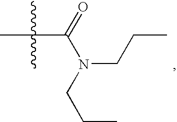

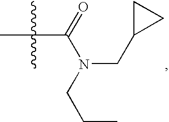

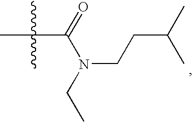

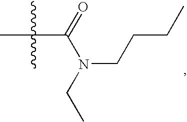

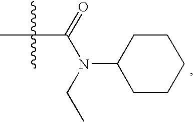

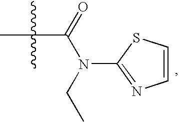

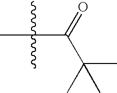

TABLE 8

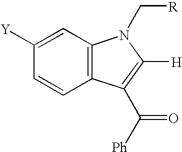

Y = OCH₃, Cl, Br, CH₂CH₃, or CN

R is:

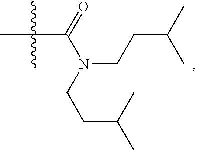

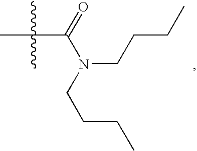

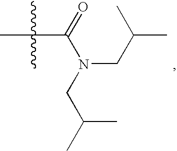

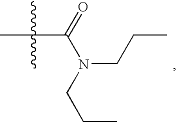

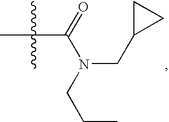

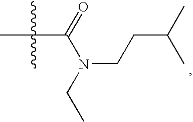

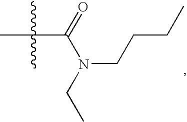

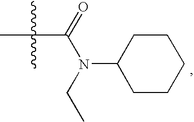

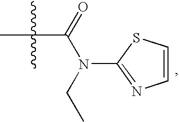

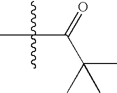

TABLE 9

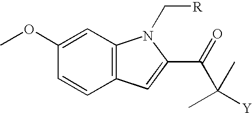

Y = CH₃ or CH₂CH₃

R is:

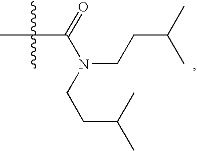

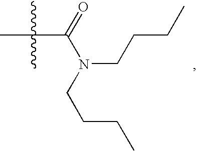

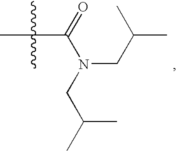

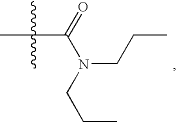

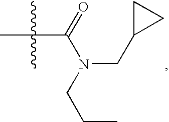

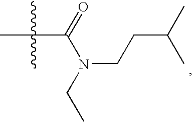

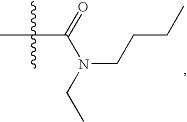

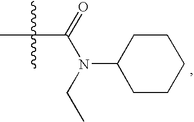

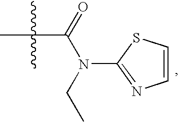

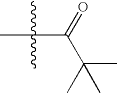

TABLE 10

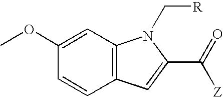

Y = OCH₃, CN, or Cl; X = H, or F; Z = Ph, CH(CH₃)₂, CH₂CH(CH₃)₂

R is:

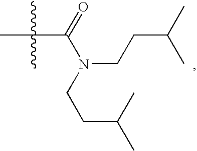

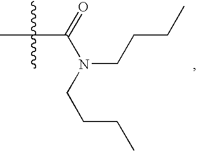

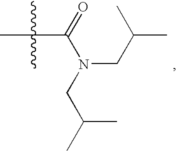

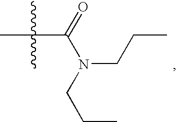

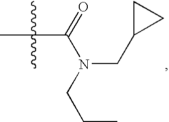

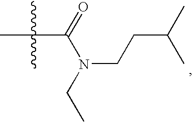

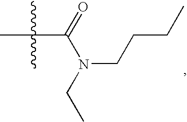

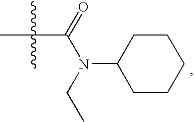

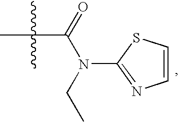

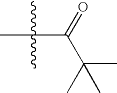

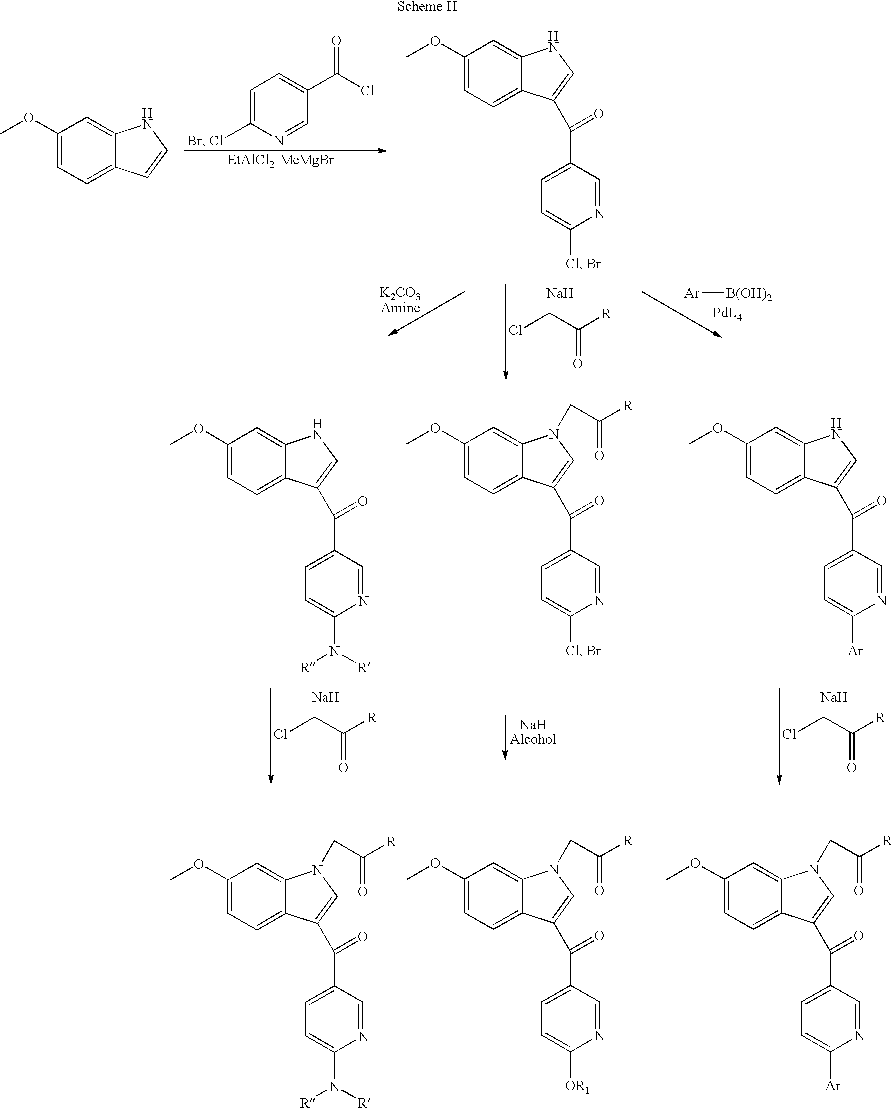

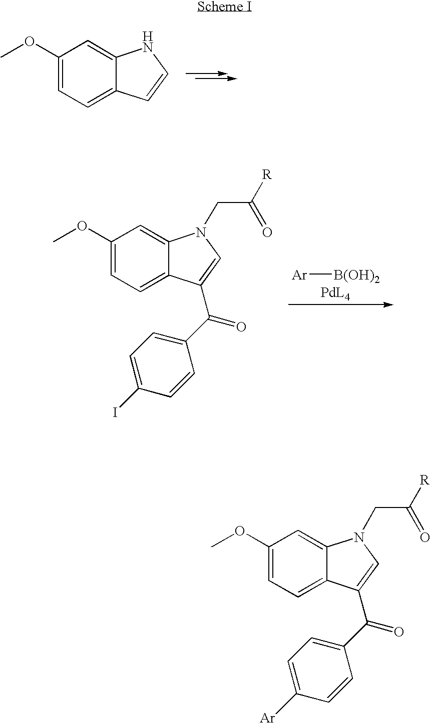

Using scheme H and I, the compounds in table 11 and 12 were prepared.

TABLE 11

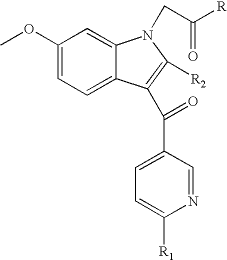

Wherein R represents:

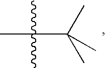

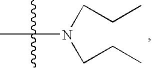

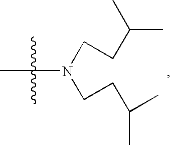

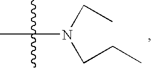

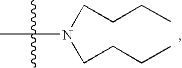

R₁ represents:

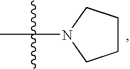

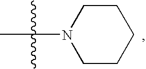

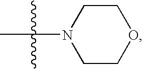

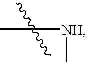

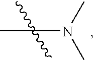

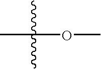

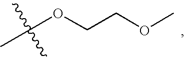

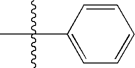

R2 represents: hydrogen or methyl

TABLE 12

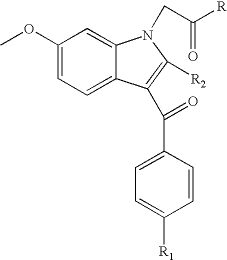

Wherein R represents:

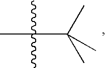

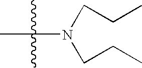

R₁ represents:

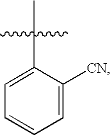

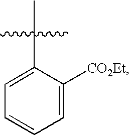

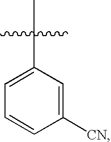

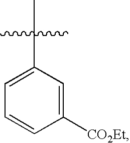

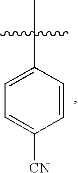

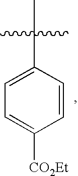

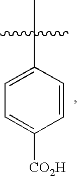

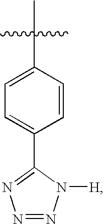

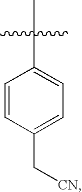

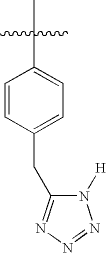

R2 represents: hydrogen or methyl

### Functional Assays

A. Maxi-K Channel

The activity of the compounds can also be quantified by the following assay.

The identification of inhibitors of the Maxi-K channel is based on the ability of expressed Maxi-K channels to set cellular resting potential after transfection of both alpha and beta1 subunits of the channel in HEK-293 cells and after being incubated with potassium channel blockers that selectively eliminate the endogenous potassium conductances of HEK-293 cells. In the absence of maxi-K channel inhibitors, the transfected HEK-293 cells display a hyperpolarized membrane potential, negative inside, close to E_{K} (−80 mV) which is a consequence of the activity of the maxi-K channel. Blockade of the Maxi-K channel by incubation with maxi-K channel blockers will cause cell depolarization. Changes in membrane potential can be determined with voltage-sensitive fluorescence resonance energy transfer (FRET) dye pairs that use two components, a donor coumarin (CC₂DMPE) and an acceptor oxanol (DiSBAC₂(3)).

Oxanol is a lipophilic anion and distributes across the membrane according to membrane potential. Under normal conditions, when the inside of the cell is negative with respect to the outside, oxanol is accumulated at the outer leaflet of the membrane and excitation of coumarin will cause FRET to occur. Conditions that lead to membrane depolarization will cause the oxanol to redistribute to the inside of the cell, and, as a consequence, to a decrease in FRET. Thus, the ratio change (donor/acceptor) increases after membrane depolarization, which determines if a test compound actively blocks the maxi-K channel.

The HEK-293 cells were obtained from the American Type Culture Collection, 12301 Parklawn Drive, Rockville, Md., 20852 under accession number ATCC CRL-1573. Any restrictions relating to public access to the microorganism shall be irrevocably removed upon patent issuance. Transfection of the alpha and beta1 subunits of the maxi-K channel in HEK-293 cells was carried out as follows: HEK-293 cells were plated in 100 mm tissue culture treated dishes at a density of 3×10⁶ cells per dish, and a total of five dishes were prepared. Cells were grown in a medium consisting of Dulbecco's Modified Eagle Medium (DMEM) supplemented with 10% Fetal Bovine serum, 1× L-Glutamine, and 1× Penicillin/Streptomycin, at 37° C., 10% CO₂. For transfection with Maxi-K hα(pCIneo) and Maxi-K hβ1(pIRESpuro) DNAs, 150 μl FuGENE™ was added dropwise into 10 ml of serum free/phenol-red free DMEM and allowed to incubate at room temperature for 5 minutes. Then, the FuGENE6™ solution was added dropwise to a DNA solution containing 25 μg of each plasmid DNA, and incubated at room temperature for 30 minutes. After the incubation period, 2 ml of the FuGENE6™/DNA solution was added dropwise to each plate of cells and the cells were allowed to grow two days under the same conditions as described above. At the end of the second day, cells were put under selection media which consisted of DMEM supplemented with both 600 μg/ml G418 and 0.75 μg/ml puromycin. Cells were grown until separate colonies were formed. Five colonies were collected and transferred to a 6 well tissue culture treated dish. A total of 75 colonies were collected. Cells were allowed to grow until a confluent monolayer was obtained. Cells were then tested for the presence of maxi-K channel alpha and beta1 subunits using an assay that monitors binding of ¹²⁵I-iberiotoxin-D19Y/Y36F to the channel. Cells expressing ¹²⁵I-iberiotoxin-D19Y/Y36F binding activity were then evaluated in a functional assay that monitors the capability of maxi-K channels to control the membrane potential of transfected HEK-293 cells using fluorescence resonance energy transfer (FRET) ABS technology with a VIPR instrument. The colony giving the largest signal to noise ratio was subjected to limiting dilution. For this, cells were resuspended at approximately 5 cells/ml, and 200 μl were plated in individual wells in a 96 well tissue culture treated plate, to add ca. one cell per well. A total of two 96 well plates were made. When a confluent monolayer was formed, the cells were transferred to 6 well tissue culture treated plates. A total of 62 wells were transferred. When a confluent monolayer was obtained, cells were tested using the FRET-functional assay. Transfected cells giving the best signal to noise ratio were identified and used in subsequent functional assays.

For functional assays:

The transfected cells (2E+06 Cells/mL) are then plated on 96-well poly-D-lysine plates at a density of about 100,000 cells/well and incubated for about 16 to about 24 hours. The medium is aspirated of the cells and the cells washed one time with 100 μl of Dulbecco's phosphate buffered saline (D-PBS). One hundred microliters of about 9 μM coumarin (CC₂DMPE)-0.02% pluronic-127 in D-PBS per well is added and the wells are incubated in the dark for about 30 minutes. The cells are washed two times with 100 μl of Dulbecco's phosphate-buffered saline and 100 μl of about 4.5 μM of oxanol (DiSBAC₂(3)) in (mM) 140 NaCl, 0.1 KCl, 2 CaCl₂, 1 MgCl₂, 20 Hepes-NaOH, pH 7.4, 10 glucose is added. Three micromolar of an inhibitor of endogenous potassium conductance of HEK-293 cells is added. A maxi-K channel blocker is added (about 0.01 micromolar to about 10 micromolar) and the cells are incubated at room temperature in the dark for about 30 minutes.

The plates are loaded into a voltage/ion probe reader (VIPR) instrument, and the fluorescence emission of both CC₂DMPE and DiSBAC₂(3) are recorded for 10 sec. At this point, 100 μl of high-potassium solution (mM): 140 KCl, 2 CaCl₂, 1 MgCl₂, 20 Hepes-KOH, pH 7.4, 10 glucose are added and the fluorescence emission of both dyes recorded for an additional 10 sec. The ratio CC₂DMPE/DiSBAC₂(3), before addition of high-potassium solution equals 1. In the absence of maxi-K channel inhibitor, the ratio after addition of high-potassium solution varies between 1.65-2.0. When the Maxi-K channel has been completely inhibited by either a known standard or test compound, this ratio remains at 1. It is possible, therefore, to titrate the activity of a Maxi-K channel inhibitor by monitoring the concentration-dependent change in the fluorescence ratio.

The compounds of this invention were found to cause concentration-dependent inhibition of the fluorescence ratio with IC₅₀'s in the range of about 1 nM to about 20 μM, more preferably from about 10 nM to about 500 nM.

B. Electrophysiological Assays of Compound Effects on High-Conductance Calcium-Activated Potassium Channels

Methods:

Patch clamp recordings of currents flowing through large-conductance calcium-activated potassium (maxi-K) channels were made from membrane patches excised from CHO cells constitutively expressing the α-subunit of the maxi-K channel or HEK293 cells constitutively expressing both α- and β-subunits using conventional techniques (Hamill et al., 1981, Pflügers Archiv. 391, 85-100) at room temperature. Glass capillary tubing (Garner #7052 or Drummond custom borosilicate glass 1-014-1320) was pulled in two stages to yield micropipettes with tip diameters of approximately 1-2 microns. Pipettes were typically filled with solutions containing (mM): 150 KCl, 10 Hepes (4-(2-hydroxyethyl)-1-piperazine methanesulfonic acid), 1 Mg, 0.01 Ca, and adjusted to pH 7.20 with KOH. After forming a high resistance (>10⁹ ohms) seal between the plasma membrane and the pipette, the pipette was withdrawn from the cell, forming an excised inside-out membrane patch. The patch was excised into a bath solution containing (mM): 150 KCl, 10 Hepes, 5 EGTA (ethylene glycol bis(β-aminoethyl ether)-N,N,N′,N′-tetraacetic acid), sufficient Ca to yield a free Ca concentration of 1-5 μM, and the pH was adjusted to 7.2 with KOH. For example, 4.193 mM Ca was added to give a free concentration of 1 μM at 22° C. An EPC9 amplifier (HEKA Elektronic, Lambrect, Germany) was used to control the voltage and to measure the currents flowing across the membrane patch. The input to the headstage was connected to the pipette solution with a Ag/AgCl wire, and the amplifier ground was connected to the bath solution with a Ag/AgCl wire covered with a tube filled with agar dissolved in 0.2 M KCl. The identity of maxi-K currents was confirmed by the sensitivity of channel open probability to membrane potential and intracellular calcium concentration.

Data acquisition was controlled by PULSE software (HEKA Elektronic) and stored on the hard drive of a MacIntosh computer (Apple Computers) for later analysis using PULSEFIT (HEKA Elektronic) and Igor (Wavemetrics, Oswego, Oreg.) software.

Results:

The effects of the compounds of the present invention on maxi-K channels was examined in excised inside-out membrane patches with constant superfusion of bath solution. The membrane potential was held at −80 mV and brief (100-200 ms) voltage steps to positive membrane potentials (typically +50 mV) were applied once per 15 seconds to transiently open maxi-K channels. As a positive control in each experiment, maxi-K currents were eliminated at pulse potentials after the patch was transiently exposed to a low concentration of calcium (<10 nM) made by adding 1 mM EGTA to the standard bath solution with no added calcium. The fraction of channels blocked in each experiment was calculated from the reduction in peak current caused by application of the specified compound to the internal side of the membrane patch. Compound was applied until a steady state level of block was achieved. K_{I} values for channel block were calculated by fitting the fractional block obtained at each compound concentration with a Hill equation. The K_{I} values for channel block by the compounds described in the present invention range from 0.01 nM to greater than 10 μM.

1. A compound of the structural formula I: 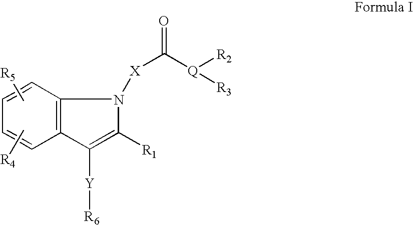
or a pharmaceutically acceptable salt, enantiomer, diastereomer or mixture thereof:
wherein,
R represents hydrogen, or C₁₋₆ alkyl;
R₁ represents hydrogen or C₁₋₆ alkyl, CF₃, C₁₋₆ alkoxy, COR^{c}, CO₂R₈, CONHCH₂CO₂R, N(R)₂, said alkyl and alkoxy optionally substituted with 1-3 groups selected from R^{b};
X represents —(CHR₇)ₚ—;
Y is not present, —CO(CH₂)ₙ—, or —CH(OR)—;
Q represents N, CR^{y}, or O, wherein R₂ is absent when Q is O;
R^{y} represents H, or C₁₋₆ alkyl;
R_{w} represents H, C₁₋₆ alkyl, —C(O)C₁₋₆ alkyl, —C(O)OC₁₋₆ alkyl, —SO₂N(R)₂, —SO₂C₁₋₆ alkyl, —SO₂C₆₋₁₀ aryl, NO₂, CN or —C(O)N(R)₂;
R₂ represents hydrogen, C₁₋₁₀ alkyl, C₁₋₆ alkylSR, —(CH₂)ₙO(CH₂)ₘOR, —(CH₂)ₙC₁₋₆ alkoxy, —(CH₂)ₙC₃₋₈ cycloalkyl, —(CH₂)ₙC₃₋₁₀ heterocyclyl, —(CH₂)ₙC₅₋₁₀ heteroaryl, —N(R)₂, —COOR, or —(CH₂)ₙC₆₋₁₀ aryl, said alkyl, heterocyclyl, aryl or heteroaryl optionally substituted with 1-3 groups selected from R^{a};
R₃ represents hydrogen, C₁₋₁₀ alkyl, —(CH₂)ₙC₃₋₈ cycloalkyl, —(CH₂)ₙC₃₋₁₀ heterocyclyl, —(CH₂)ₙC₅₋₁₀ heteroaryl, —(CH₂)ₙCOOR, —(CH₂)ₙC₆₋₁₀ aryl, —(CH₂)ₙNHR₈, —(CH₂)ₙN(R)₂, —(CH₂)ₙNHCOOR, —(CH₂)ₙN(R₈)CO₂R, —(CH₂)ₙN(R₈)COR, —(CH₂)ₙNHCOR, —(CH₂)ₙCONH(R₈), aryl, —(CH₂)ₙC₁₋₆ alkoxy, CF₃, (CH₂)ₙSO₂R, —(CH₂)ₙSO₂N(R)₂, —(CH₂)ₙCON(R)₂, —(CH₂)ₙCONHC(R)₃, —(CH₂)ₙCOR₈, nitro, cyano or halogen, said alkyl, alkoxy, heterocyclyl, aryl or heteroaryl optionally substituted with 1-3 groups of R^{a};
or, when Q is N, R₂ and R₃ taken together with the intervening N atom form a 4-10 membered heterocyclic carbon ring optionally interrupted by 1-2 atoms of O, S, C(O) or NR, and optionally having 1-4 double bonds, and optionally substituted by 1-3 groups selected from R^{a};
R⁴ and R⁵ independently represent hydrogen, C₁₋₆ alkoxy, OH, C₁₋₆ alkyl, COOR, SO₃H, O(CH₂)ₙN(R)₂, O(CH₂)ₙCO₂R, C₁₋₆ alkylcarbonyl, S(O)qR^{y}, OPO(OH)₂, CF₃, N(R)₂, nitro, cyano or halogen;
R₆ represents hydrogen, C₁₋₁₀ alkyl, —(CH₂)ₙC₆₋₁₀ aryl, —(CH₂)ₙC₅₋₁₀ heteroaryl, (C₆₋₁₀ aryl)O—, —(CH₂)ₙC₃₋₁₀ heterocyclyl, —(CH₂)ₙC₃₋₈ cycloalkyl, —COOR, —C(O)CO₂R, said aryl, heteroaryl, heterocyclyl and alkyl optionally substituted with 1-3 groups selected from R^{a};
R⁷ represents hydrogen, C₁₋₆ alkyl, —(CH₂)ₙCOOR or —(CH₂)ₙN(R)₂,
R⁸ represents —(CH₂)ₙC₃₋₈ cycloalkyl, —(CH₂)_{n 3-10} heterocyclyl, C₁₋₆ alkoxy or —(CH₂)ₙC₅₋₁₀ heteroaryl, said heterocyclyl, aryl or heteroaryl optionally substituted with 1-3 groups selected from R^{a};
R^{a} represents F, Cl, Br, I, CF₃, N(R)₂, NO₂, CN, —COR₈, —CONHR₈, —CON(R₈)₂, —O(CH₂)ₙCOOR, —NH(CH₂)ₙOR, —COOR, —OCF₃, —NHCOR, —SO₂R, —SO₂NR₂, —SR, (C₁-C₆ alkyl)O—, —(CH₂)ₙO(CH₂)ₘOR, —(CH₂)ₙC₁₋₆ alkoxy, (aryl)O—, —OH, (C₁-C₆ alkyl)S(O)ₘ—, H₂N—C(NH)—, (C₁-C₆ alkyl)C(O)—, (C₁-C₆ alkyl)OC(O)NH—, —(C₁-C₆ alkyl)NR_{w}(CH₂)ₙC₃₋₁₀ heterocyclyl-R_{w}, —(C₁-C₆ alkyl)O(CH₂)ₙC₃₋₁₀ heterocyclyl-R_{w}, —(C₁-C₆ alkyl)S(CH₂)ₙC₃₋₁₀ heterocyclyl-R_{w}, —(C₁-C₆ alkyl)-C₃₋₁₀ heterocyclyl-R_{w}, —(CH₂)ₙ-Z¹-C(=Z²)N(R)₂, —(C₂₋₆ alkenyl)NR_{w}(CH₂)ₙC₃₋₁₀ heterocyclyl-R_{w}, —(C₂₋₆ alkenyl)O(CH₂)ₙC₃₋₁₀ heterocyclyl-R_{w}, —(C₂₋₆ alkenyl)S(CH₂)ₙC₃₋₁₀ heterocyclyl-R_{w}, —(C₂₋₆ alkenyl)-C₃₋₁₀ heterocyclyl-R_{w}, —(C₂₋₆ alkenyl)-Z¹-C(=Z²)N(R)₂, —(CH₂)ₙSO₂R, —(CH₂)ₙSO₃H, —(CH₂)ₙPO(OR)₂, cyclohexyl, morpholinyl, piperidyl, pyrrolidinyl, thiophenyl, phenyl, pyridyl, imidazolyl, oxazolyl, isoxazolyl, thiazolyl, thienyl, furyl, isothiazolyl, C₂₋₆ alkenyl, and C₁-C₁₀ alkyl, said alkyl, alkenyl, alkoxy, phenyl, pyridyl, imidazolyl, oxazolyl, isoxazolyl, thiazolyl, thienyl, furyl, and isothiazolyl optionally substituted with 1-3 groups selected from C₁-C₆ alkyl, CN, (CH₂)ₙtetrazolyl, COOR, SO₃H, OH, F, Cl, Br, I, —O(CH₂)ₙCH(OH)CH₂SO₃H, and 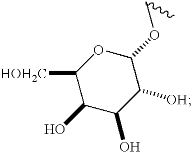
Z¹ and Z² independently represents NR_{w}, O, CH₂, or S;
R^{b} represents C₁₋₆ alkyl, —COOR, —SO₃R, —OPO(OH)₂, —(CH₂)ₙC₆₋₁₀ aryl, or —(CH₂)ₙC₅₋₁₀ heteroaryl;
R^{c} represents hydrogen, C₁₋₆ alkyl, or —(CH₂)ₙC₆₋₁₀ aryl;
m is 0-3;
n is 0-3;
q is 0-2; and
p is0-1.

2. A compound of the structural formula I wherein X represents CHR₇.

3. A compound according to claim 1 wherein Y is —CO(CH₂)ₙ.

4. A compound according to claim 1 wherein Y is CH(OR).

5. A compound according to claim 1 wherein Q is N.

6. A compound according to claim 1 wherein Q is CH.

7. A compound according to claim 2 wherein R₆ is (CH₂)ₙC₆₋₁₀ aryl, (CH₂)ₙC₅₋₁₀ heteroaryl, (CH₂)ₙC₃₋₁₀ heterocyclyl, or (CH₂)ₙC₃₋₈ cycloalkyl, said aryl, heteroaryl, heterocyclyl and alkyl optionally substituted with 1 to 3 groups of R^{a}.

8. A compound according to claim 6 wherein R₇ is hydrogen or C₁₋₆ alkyl.

9. A compound according to claim 6 wherein Q is N and n is 0.

10. A compound according to claim 1 wherein Y is —CO(CH₂)ₙ, Q is N, n is 0, R₂ is C₁₋₁₀ alkyl or C₁₋₆ alkylOH and R₃ is (CH₂)ₙC₃₋₁₀ heterocyclyl, said heterocyclyl and alkyl optionally substituted with 1 to 3 groups of R^{a}.

11. A compound selected from Tables 1 through 14 which is:
TABLE 1 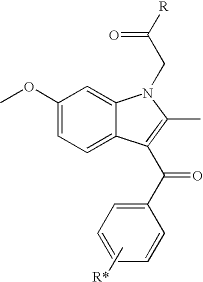
Wherein R represents: 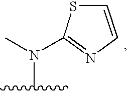 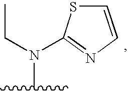 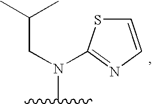 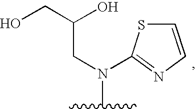
and R* represents: 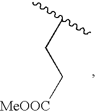 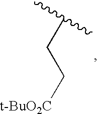 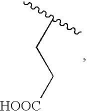 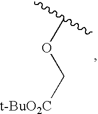 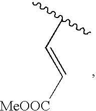 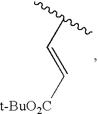 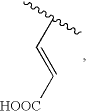 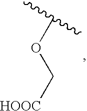
or hydrogen;
TABLE 2 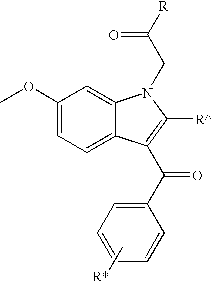
Wherein R represents: 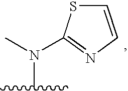 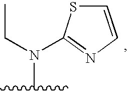 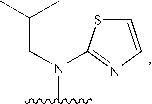 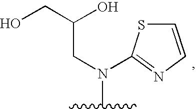
R* represents: 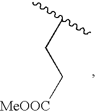 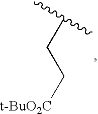 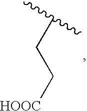 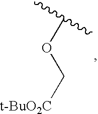 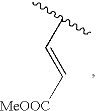 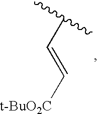 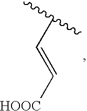 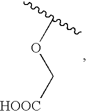
or hydrogen and R\hat{ } represents hydrogen or methyl;
TABLE 3 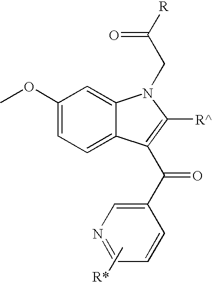
Wherein R represents: 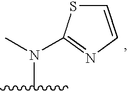 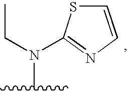 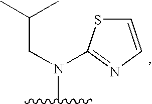 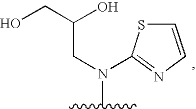 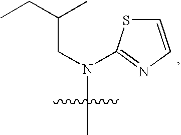
R* represents: 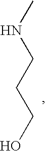
or chlorine
and R\hat{ } represents hydrogen or methyl;
TABLE 4 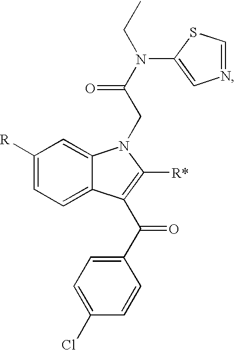
R represents methyl or methoxy and R* represents methyl, H or COCH; 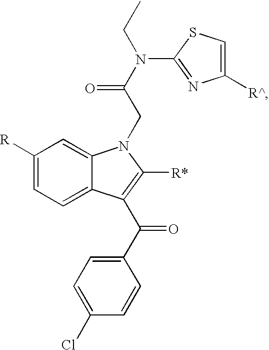
R′ represents methyl or methoxy;
R\hat{ } represents hydrogen or COOEt;
R′′′ represents COOH or COOtBu; a
R″ represents: COOMe, H, COOH, or 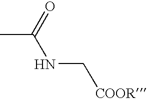
TABLE 5 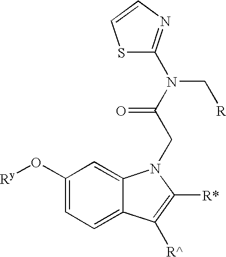
R* represents hydrogen or methyl;
R^{y} represents methyl or CF₃; 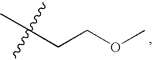 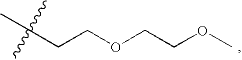 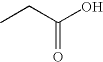
R represents methyl, (CH2)₂SCH₃, 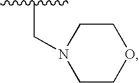 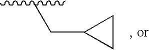 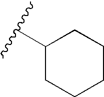
R\hat{ } represents: 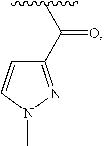 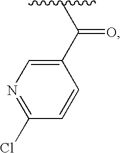 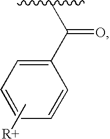 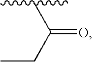 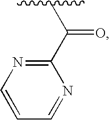 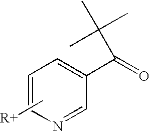
R+ represents:
(CH₂)₂CO₂H, chlorine, 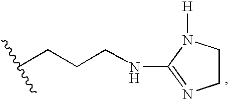 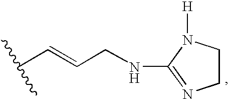 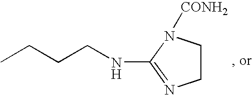 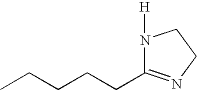
TABLE 6 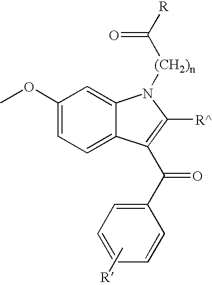 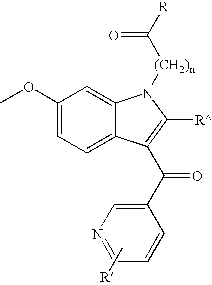
Wherein n represents 1-2;
R\hat{ } represents hydrogen or methyl
R represents: 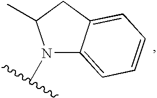 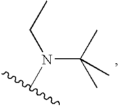 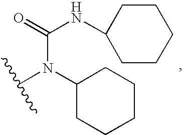 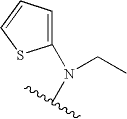 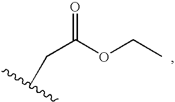 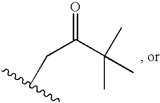 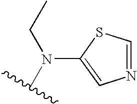
and R′ represents:
chlorine, 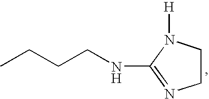 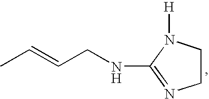 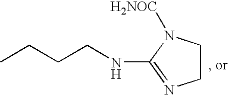 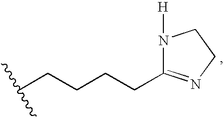
TABLE 7 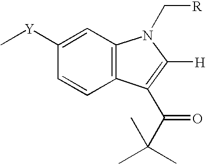
Y = O, or S(O)R and p = 0-2
R is: 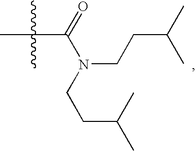 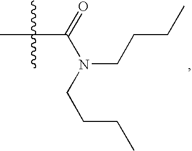 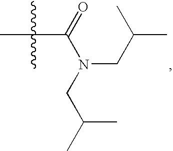 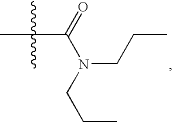 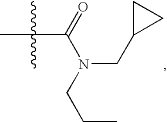 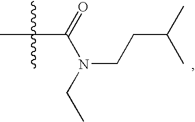 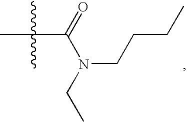 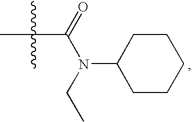 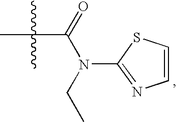 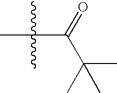
TABLE 8 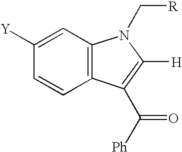
Y = OCH₃, Cl, Br, CH₂CH₃, or CN
R is: 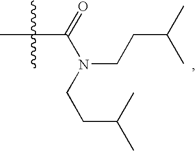 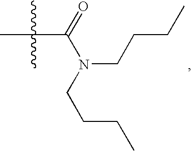 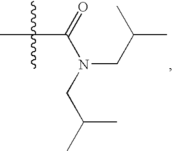 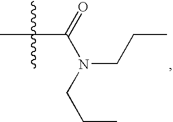 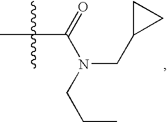 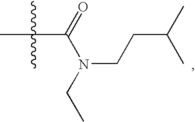 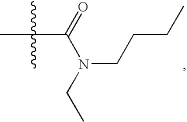 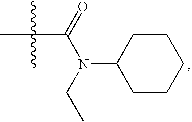 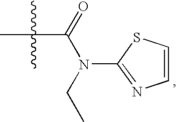 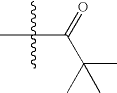
TABLE 9 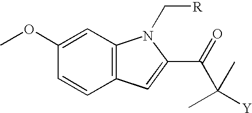
Y = CH₃ or CH₂CH₃
R is: 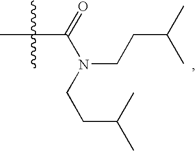 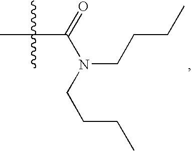 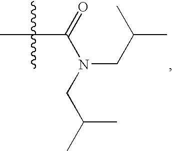 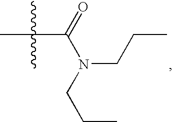 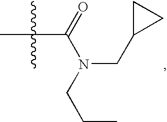 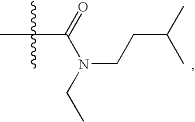 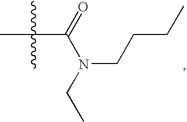 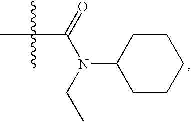 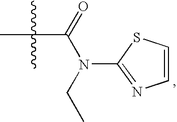 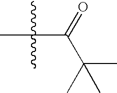
TABLE 10 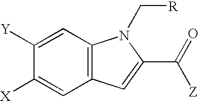
Y = OCH₃, CN, or Cl; X = H, or F;
Z = Ph, CH(CH₃)₂, CH₂CH(CH₃)₂
R is: 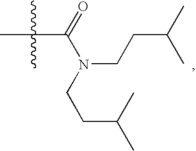 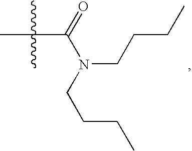 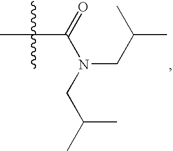 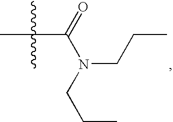 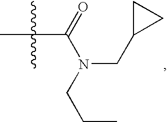 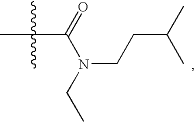 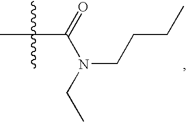 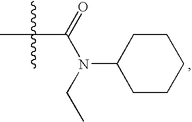 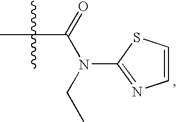 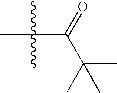
TABLE 11 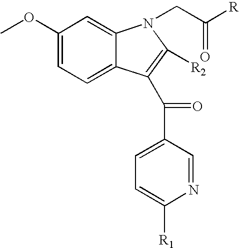
Wherein R represents:

| | | |
|---|---|---|
| 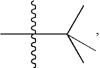 | 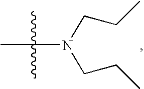 | 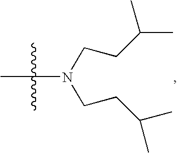 |
| | |
|---|---|
| 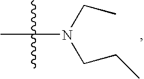 | 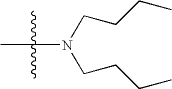 |

R₁ represents:

| | | |
|---|---|---|
| 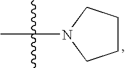 | 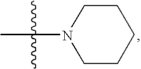 | 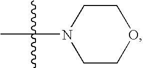 |
| | | |
|---|---|---|
| 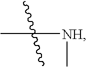 | 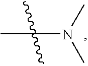 | 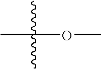 |
| | |
|---|---|
| 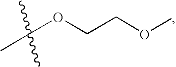 | 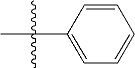 |

R2 represents: hydrogen or methyl
TABLE 12 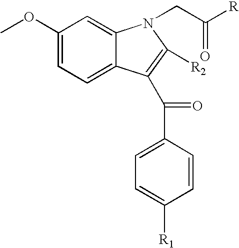
Wherein R represents:

| | |
|---|---|
| 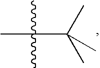 | 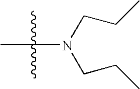 |

R₁ represents:

| | | |
|---|---|---|
| 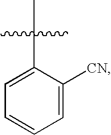 | 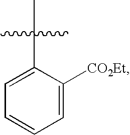 | 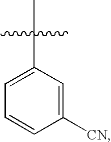 |
| | | |
|---|---|---|
| 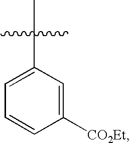 | 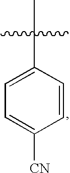 | 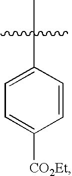 |
| | | |
|---|---|---|
| 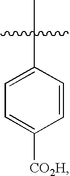 | 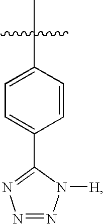 | 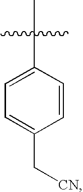 |

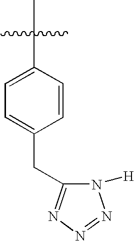

R2 represents: hydrogen or methyl
TABLE 13 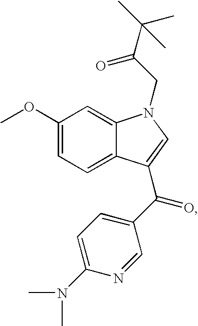 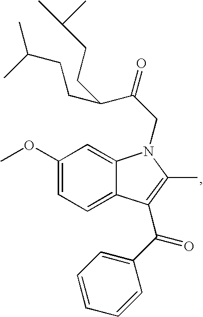 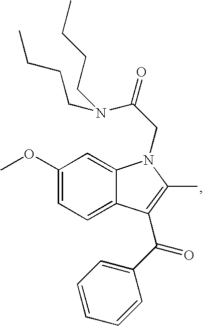 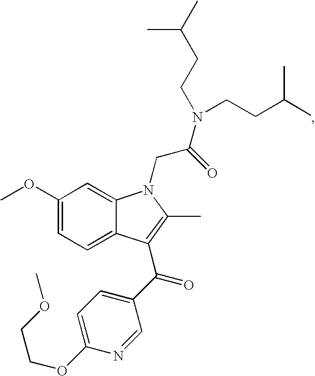 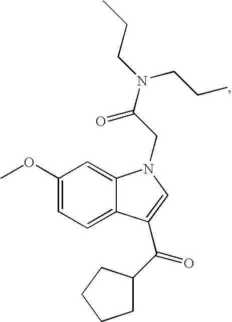 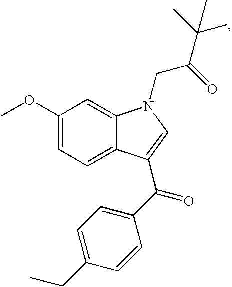 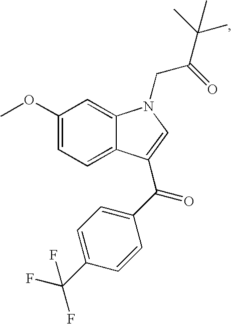 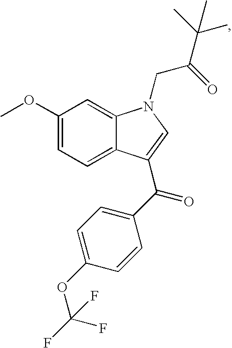 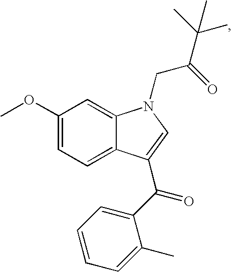 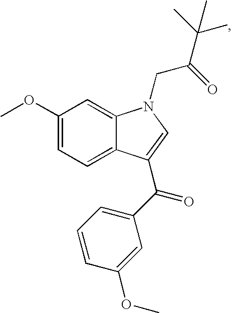 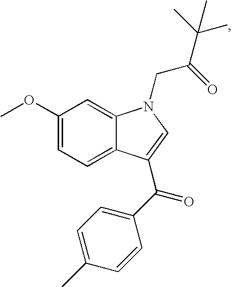 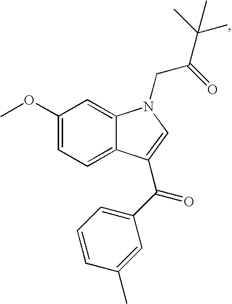 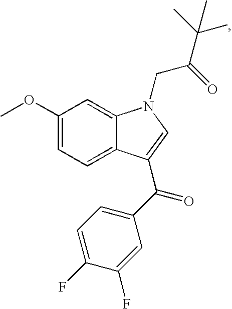 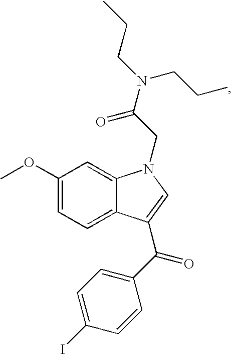 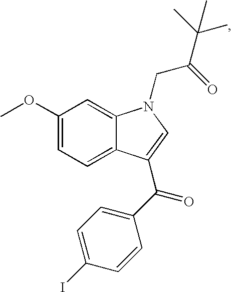 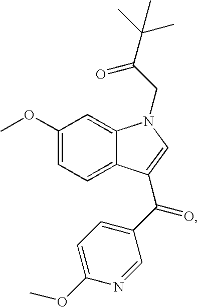 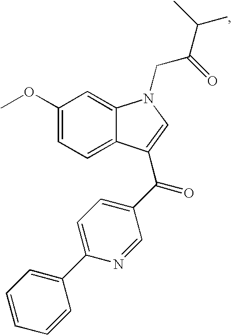 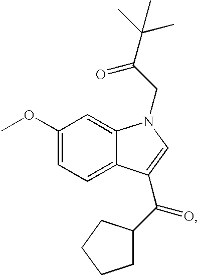
TABLE 14 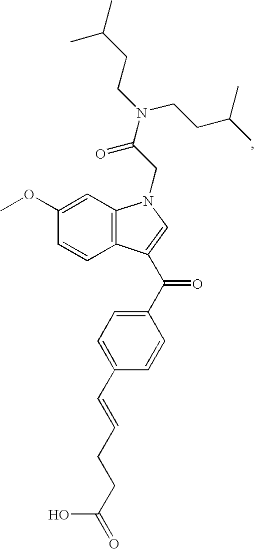 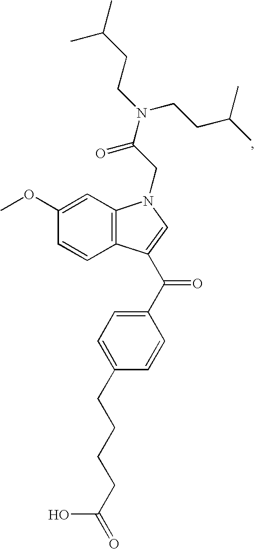 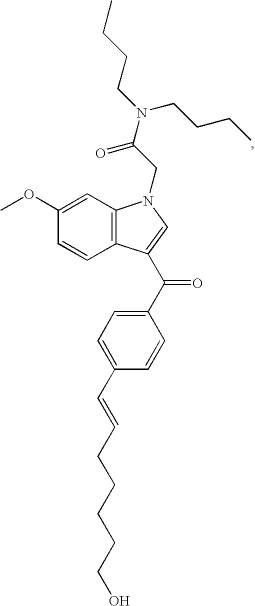 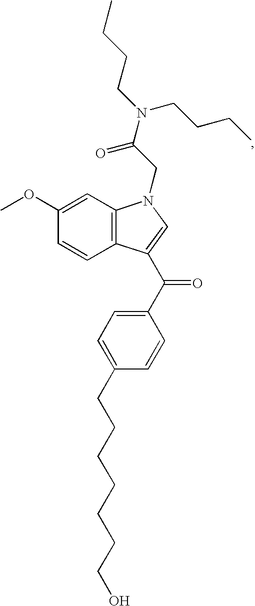 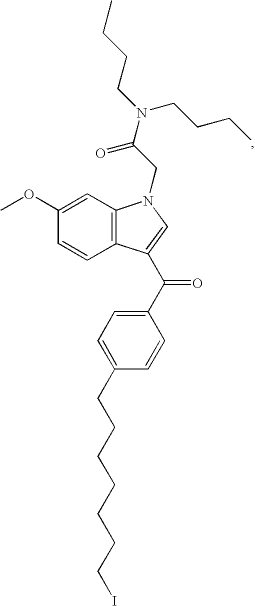 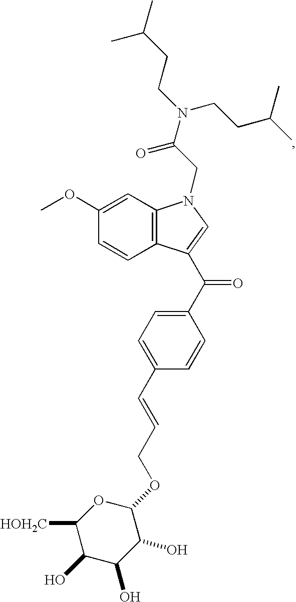 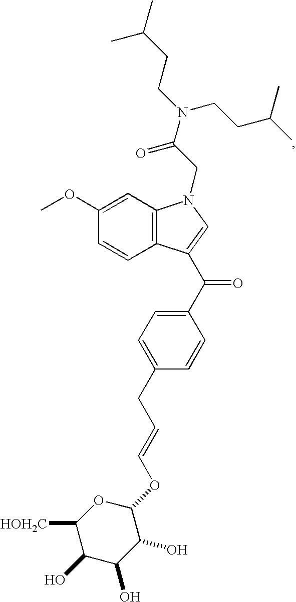 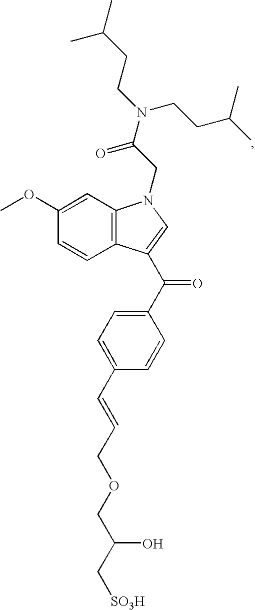 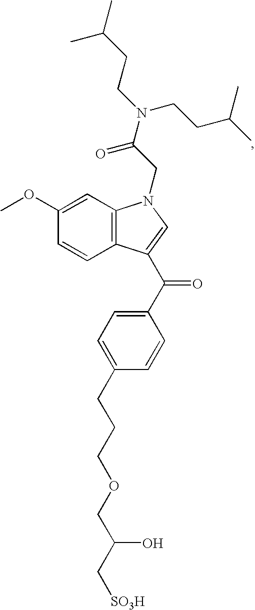 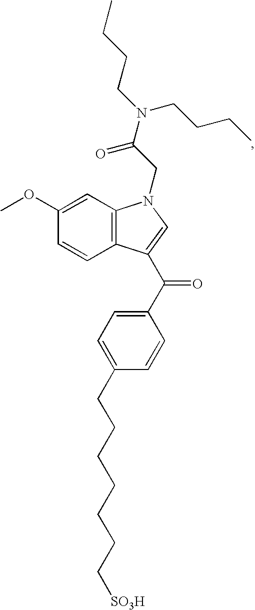 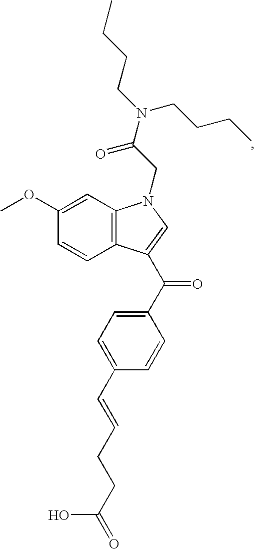 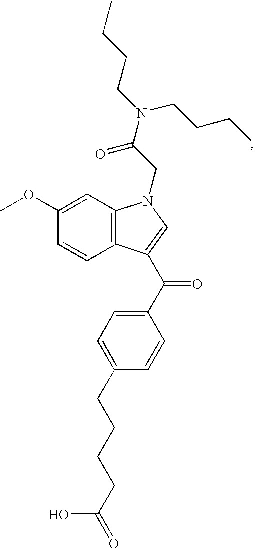 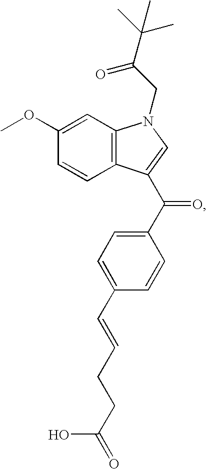 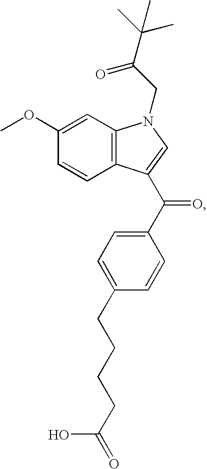
or a pharmaceutically acceptable salt, enantiomer, diastereomer or mixture thereof.

12. A method for treating ocular hypertension or glaucoma comprising administration to a patient in need of such treatment a therapeutically effective amount of a compound of claim 1.

13. The method according to claim 12 wherein the compound of formula I is applied as a topical formulation selected from solution topical formulation and a suspension topical formulation.

14. A method according to claim 13 in which the topical formulation optionally contains xanthan gum or gellan gum.

15. A method according to claim 13 wherein an active ingredient belonging to the group consisting of: δ-adrenergic blocking agent, parasympathomimetic agent, EP4 agonist, carbonic anhydrase inhibitor, and a prostaglandin or a prostaglandin derivative is optionally added to the formulation.

16. A method according to claim 15 wherein the δ-adrenergic blocking agent is timolol; the parasympathomimetic agent is pilocarpine; the carbonic anhydrase inhibitor is dorzolamide, acetazolamide, metazolamide or brinzolamide; the prostaglandin is latanoprost or rescula, and the prostaglandin derivative is a hypotensive lipid derived from PGP2α prostaglandins.

17. A method for treating macular edema, macular degeneration, increasing retinal and optic nerve head blood velocity, increasing retinal and optic nerve oxygen tension, and/or providing a neuroprotective effect comprising administration to a patient in need of such treatment a pharmaceutically effective amount of a compound of claim 1; or a pharmaceutically acceptable salt, enantiomer, diastereomer or mixture thereof.

18. The method according to claim 17 wherein the compound of formula I is applied as a topical formulation.

19. A method according to claim 18 in which the topical formulation optionally contains xanthan gum or gellan gum.

20. A method of preventing repolarization or hyperpolarization of a mammalian cell wherein the cell contains a potassium channel comprising the administration to a mammal, including a human, in need thereof, of a pharmacologically effective amount of a compound according to claim 1, or a pharmaceutically acceptable salt, enantiomer, diastereomer or mixture thereof

21. A method of treating Alzheimer's Disease, depression, cognitive disorders, arrhythmia disorders and/or diabetes in a patient in need thereof comprising administering a pharmaceutically effective amount of a compound according to claim 1, or a pharmaceutically acceptable salt, enantiomer, diastereomer or mixture thereof.
